(19) United States
(12) Patent Application Publication
(10) Pub. No.: US 2024/0335527 A1
(43) Pub. Date: Oct. 10, 2024

(54) VACCINE ADJUVANTS, TRANSFECTION REAGENTS, AND METHODS OF USING THE SAME

(71) Applicant: Wisconsin Alumni Research Foundation, Madison, WI (US)

(72) Inventors: Adel Talaat, Middleton, WI (US); Bubacarr Touray, Madison, WI (US)

(21) Appl. No.: 18/629,296

(22) Filed: Apr. 8, 2024

Related U.S. Application Data
(60) Provisional application No. 63/494,684, filed on Apr. 6, 2023.

(51) Int. Cl. A61K 39/215; A61K 9/51; A61K 39/00; A61K 39/04; A61K 39/145; A61P 37/04

(52) U.S. Cl. CPC A61K 39/215; A61K 9/5123; A61K 9/5192; A61K 39/04; A61K 39/145; A61P 37/04; A61K 2039/53; A61K 2039/552; A61K 2039/55577

(57) ABSTRACT

Disclosed herein are compositions of disaggregated spherical nanostructures comprising Quil-A and dioleoyl 3 trimethylammonium propane (DOTAP) wherein the Quil-A and DOTAP are present at ratios between 2:1 Quil-A: DOTAP to about 1:2 Quil-A: DOTAP. Also provided are methods of making and using the same.

### CROSS-REFERENCE TO RELATED APPLICATIONS

The present application claims priority to U.S. Provisional Patent Application No. 63/494,684 that was filed Apr. 6, 2023, the entire contents of which are hereby incorporated by reference.

### STATEMENT REGARDING FEDERALLY SPONSORED RESEARCH

This invention was made with government support under 2020-67021-31256 awarded by the USDA/NIFA. The government has certain rights in the invention.

### BACKGROUND OF THE INVENTION

Over the past few decades, data derived from studies using nucleic acid vaccination as a mechanism of combating infectious diseases and cancers is promising. Nucleic acid vaccines address the safety concerns associated with live attenuated vaccines and their development pipeline makes them easily scalable and cost effective [1]. The COVID-19 pandemic caused by SARS-CoV-2 caused devastating effects on global health and economies with more than 300 million registered cases and more than 5.7 million deaths globally [2]. The scientific community responded by leveraging the messenger ribonucleic acid (mRNA) vaccine technology and within few months of releasing the genetic sequence of SARS-CoV-2, the first mRNA vaccine was developed and tested [3]. However, mRNA vaccines cannot be administered in their naked form because mammalian RIG-kinases rapidly degrade mRNA rendering it non-functional. To prevent degradation of the mRNA, scientists utilize technologies that can efficiently deliver mRNA to mammalian cells for protein translation [4]. Lipid Nanoparticles (LNPs) have been used as efficient nucleic acid delivery technology that has been shown to be both safe and scalable. Indeed, both FDA approved mRNA COVID-19 vaccines are encapsulated in LNPs of various formulations [5].

Furthermore, adjuvants are included in LNP formulations to enhance immunogenicity of vaccines thereby improving their efficacy [6, 7]. Live attenuated or weakened organisms as vaccines contain naturally occurring adjuvants that helps the body to produce a strong protective immune response. On the contrary, vaccines such as mRNA vaccines contain an mRNA encoding a component of the pathogen and the mRNA by itself is not able to elicit the desirable protective immune response. Thus, adjuvants are included in mRNA vaccine formulations to enhance their immunogenicity for better protective immunity [8].

Presented herein are compositions and methods for producing a lipid nanoparticle comprising cationic lipid 1,2-dioleoyl-3-trimethylammonium-propane (DOTAP) and Quil-A, a powdered saponin fraction isolated from extract of the bark of Quillaja Saponaria trees. This LNP formulation is referred to as QTAP, where “Q” represents Quil-A while “TAP” represents DOTAP.

### BRIEF SUMMARY OF THE INVENTION

Disclosed herein are compositions comprising disaggregated spherical nanostructures comprising Quil-A and 1,2-dioleoyl-3-trimethylammonium propane (DOTAP) wherein the Quil-A and DOTAP are present at ratios between 2:1 Quil-A:DOTAP to about 1:2 Quil-A:DOTAP. In some embodiments, the composition further comprises a payload molecule.

Also disclosed herein are methods of forming a composition comprising Quil-A and DOTAP spherical nanostructures, comprising the steps of: i) Diluting Quil-A in a buffer, wherein Quil-A is present at a final concentration of about 0.02% to about 0.001%; ii) diluting DOTAP in a buffer in a separate container from Quil-A, wherein DOTAP is present at a final concentration of about 0.1% to about 0.008%; iii) combining the contents of (i) with the contents of (ii) and vortexing the combination, e.g., at high speed, for at least 15 seconds and iv) incubating the combined vortexted solution of (iii) whereby a composition comprising Quil-A and DOTAP nanostructures are formed. In some embodiments the methods further comprise a payload molecule.

Another aspect of the present disclosure provides a vaccine formulation comprising the composition provided herein.

Another aspect of the present disclosure provide methods of generating an immune response in a subject, comprising the step of administering to a subject the composition or the vaccine formulation provided herein.

Another aspect of the present disclosure provides methods of delivering a payload inside a cell, comprising contacting the cell with the composition provided herein.

### BRIEF DESCRIPTION OF THE DRAWINGS

Non-limiting embodiments of the present invention will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the invention shown where illustration is not necessary to allow those of ordinary skill in the art to understand the invention.

FIGS. 1A, 1B, 1C, 1D, 1E, and IF. QTAP forms nanoparticles and releases RNA cargo in a sustained manner with high stability. (A) Electron microgram of QTAP and DOTAP-encapsulated mRNA measured by TEM. (B) DLS data of NPs measured at 25° C. with Zetasizer software. (C) In vitro sustained release kinetics of packaged mRNA measured at pH-7.4, 37° C. (D-F) Relative expression of Luc protein in BHK cells transfected with QTAP-encapsulated Luc mRNA stored at different temperatures measured by luminometry. Data were plotted in GraphPad Prism and one-way ANOVA was used to examine differences between samples.

FIGS. 2A, 2B, 2C, and 2D. QTAP nanovaccines can efficiently deliver modified mRNA leading to higher protein expression in cells. (A and B) Relative expression of Luc protein in BHK cells transfected with QTAP-encapsulated Luc mRNA determined by luminometry. (C) Fluorescence microscope image of GFP expression in BHK cells transfected with either DOTAP or QTAP-encapsulated GFP mRNA. (D) Transfection efficiency of DOTAP and QTAP-encapsulated GFP mRNA in BHK cells measured by flow cytometry. Data was analyzed and plotted in GraphPad Prism and statistical differences calculated by One-way ANOVA. Asterisks indicate statistical significance, where *p<0.05, **p<0.01, ***p<0.001, and ****p<0.0001.

FIGS. 3A, 3B, 3C, and 3D. QTAP encapsulating mRNA-Ag85B is not cytotoxic and activates macrophages. (A) Protein expression of modified vs non-modified mRNA determined by Western blot analysis using anti-histidine antibody lane 1-10: Ladder, negative control, GFP, modified Ag85B, modified Hsp70, modified GFP, unmodified Ag85B, unmodified Hsp70, positive control (SARS-Cov-2 histidine-tagged Nucleocapsid protein). (B, C) Macrophages transfected with QTAP-Ag85B mRNA were used to examine the cellular expression of macrophage activation markers CD80 and CD86 measured by flow cytometry. (D) Activation of macrophage inflammatory pathways in response to QTAP-Ag85B mRNA stimulation determined by One-step qRT-PCR using gene-specific primers. (E, F) BHK and J774 cells were transfected with QTAP-mRNA-Ag85B (1 ug) for 24 hr after which viability was determined by MTT cytotoxicity assay. All data were analyzed and plotted in GraphPad Prism and statistical differences were calculated by One-way ANOVA. The black line and error bars show mean+SD. Asterisks indicate statistical significance, where *p<0.05, **p<0.01, ***p<0.001, and ****p<0.0001.

FIGS. 4A, 4B, 4C, 4D, and 4E. QTAP-mRNA is both safe and immunogenic in mice. (A) Immunogenicity of QTAP encapsulating 15 ug of mRNA (Ag85B+Hsp70) was determined in 2-week-old C57Bl/6 mice using three-dose vaccination at 5-week intervals. (B) Mouse weight was measured every week and presented as mean+/−standard deviation. Serum from vaccinated mice collected after the final vaccine dose and used for cytokine ELISA targeting IFN-γ (C), TNF-α (D), and IL-17 (E). All data were analyzed and plotted in GraphPad Prism and statistical differences were calculated by One-way ANOVA. Dots represent individual mice (n=10-14/group) and the black line and error bars show mean+SD. Asterisks indicate statistical significance, where *p<0.05, **p<0.01, ***p<0.001, and ****p<0.0001.

FIGS. 5A, 5B, 5C, 5D, 5E, and 5F. Immunization of mice with QTAP-mRNA Ag85B+Hsp70 is safe and elicits a robust T cell immune response. (A-D) Lung cells harvested after the final vaccine dose were stimulated with PPD ex vivo and evaluated for CD4 T cell responses by intracellular cytokine staining flow cytometry. Percent frequency of CD4⁺ PPD specific cytokine-producing cells four-weeks post final immunization. Serums collected at 5 weeks after the second vaccine dose (E) and four-week post-challenge (F) were analyzed for IgG response by ELISA. Data were analyzed by FlowJo and GraphPad prism used for one-way ANOVA analysis. Dots represent individual mice (n=3/group) and the black line and error bars show mean±SD. Asterisks indicate statistical significance, where *p<0.05, **p<0.01, ***p<0.001, and ****p<0.0001.

FIGS. 6A, 6B, 6C, 6D, 6E, and 6F. Immunization of mice with QTAP-mRNA Ag85B+Hsp70 is protective against M. ah infection for up to 8 weeks. C57BL/6 mice were infected with M. ah by aerosol route four weeks post final immunization. (A-D) Bacterial burden was determined by colony forming unit (CFU) in the lung (A and C) and spleen (B and D) four-(A and B) and eight-weeks (C and D) post-challenge. Dots represent individual mice (n=4-5/group) and the black line and error bars show mean±SD. Histological assessment was done by H&E staining of lung tissue sections at 4 weeks (E) and 8 weeks (F) post-challenge. Arrowheads represent granuloma-like structures. Scale bars represent magnification (5×). Asterisks indicate statistical significance, where *p<0.05, **p<0.01, ***p<0.001, and ****p<0.0001.

FIGS. 7A, 7B, 7C, 7D, 7E, 7F, 7G, 7H, and 7I. Mice immunized with QTAP-mRNA Ag85B+Hsp70 elicit a robust immune in the lungs upon infection with M. ah up to 8 weeks. C57BL/6 mice were infected with M. ah by aerosol route four weeks post final immunization. After four- and eight-weeks post-challenge, mice were euthanized for lung and spleen and were evaluated for CD4 T cell responses by intracellular cytokine staining flow cytometry. (A-D) Percent frequency of CD4⁺ PPD specific cytokine-producing cells at four weeks post-challenge. (E-H) Percent frequency of CD4⁺ PPD specific cytokine-producing cells at eight weeks post-challenge. (I) Percent frequency of CD4⁺ PPD specific cytokine-producing IFN-γ, IL-2, and TNF-α cells at eight weeks post-challenge. Data were analyzed by FlowJo and GraphPad prism used for one-way ANOVA analysis. Dots represent individual mice (n=4-5/group) and the black line and error bars show mean+SD. Asterisks indicate statistical significance, where *p<0.05, **p<0.01, ***p<0.001, and ****p<0.0001.

FIGS. 8A and 8B. shows (A) fluorescent microscopy image of BHK cells transfected with either a commercial transfection reagent or the QTAP nanoadjuvant, and (B) SDS page showing western blot findings of hemagglutinin (HA) protein detected by HA specific antibody in BHK cells transfected with either QTAP nanoadjuvant or commercial transfected reagents.

FIGS. 9A and 9B show (A) a schematic of the experimental method and (B) weekly weight of chicks administered the disclosed compositions comprising the indicated payloads: phosphate buffered saline (PBS), naked influenza hemagglutinin (HA) DNA (mHA), or Quil-A chitosan encapsulated DNA vaccine encoding HA, administered in two doses as shown in 9A, SQ=subcutaneous route of administration and ON=Oculo-nasal route of administration.

FIGS. 10A, 10B, 10C, and 10D show influenza hemagglutinin inhibition titers determined from chicken sera in the experiments outlined in FIG. 9A. A-D, day 14, 21, 28, and 35 post-vaccinations.

### DETAILED DESCRIPTION OF THE INVENTION

The present invention provides a highly efficient transfection agent, termed QTAP, which can elicit an immune response in a subject, improve the immunogenicity of vaccines, as well as methods of making and using the same. QTAP is a nanoadjuvant system compromising Quil-A and DOTAP (1,2-dioleoyl-3-trimethylammonium propane), which are combined to form the portmanteau “QTAP.” QTAP has been demonstrated to be useful for the efficient delivery of mRNA vaccines constructs into cells. The data described herein show that complexation of mRNA with QTAP forms nanoparticles with average size of 75 nm and have ˜90% encapsulation efficiency, by electron microscopy. Incorporation of pseudouridine modified mRNA resulted in higher transfection efficiency and protein translation with lower cytotoxicity than unmodified mRNA. When QTAP-mRNA or QTAP alone was transfected into macrophages, pro-inflammatory pathways (e.g., NLRP3, NF-k, and MyD88) were upregulated, an indication of macrophage activation and an elicitation of an immune response. In C57Bl/6 mice, QTAP nanovaccines encoding Ag85B and Hsp70 transcripts (QTAP-85B+H70) were able to elicit robust proinflammatory responses dominated by IFN-γ, TNF-α, IL-2, and IL-17 cytokines. Following aerosol challenge with a clinical isolate of M. avium ss. hominissuis (M. ah), significant reduction of mycobacterial counts was observed in lungs and spleens of only immunized animals at both 4- and 8-weeks post challenge. As expected, reduced levels of M. ah were associated with diminished histological lesions and robust cell-mediated immunity. Interestingly, polyfunctional T-cells expressing IFN-γ, IL-2, and TNF-α were detected at 8 but not 4 weeks post challenge.

### Compositions

One aspect of the present invention comprises a composition of disaggregated spherical nanostructures comprising Quil-A and dioleoyl 3 trimethylammonium propane (DOTAP). Quil-A is a highly purified and concentrated saponin adjuvant purified from bark extract of the Quillaja saponaria Molina tree which has been specifically developed for use as an adjuvant in veterinary vaccines. Quil-A consists of a complex mixture of approximately 25 different saponin molecules which have the triterpenoid backbone in common. It is a very versatile adjuvant due to its ability to induce a balanced immune response to protect against both intracellular and extracellular pathogens.

1,2-dioleoyl-3-trimethylammonium propane (DOTAP) (also known as 18:1TAP) is a cationic surfactant. DOTAP is used as an active ingredient in fabric softeners and as a liposomal-transfection agent for DNA, RNA or other negatively charged molecules. DOTAP is a biodegradable analogue of 1,2-di-O-octadecenyl-3-trimethylammonium-propane (DOTMA) and can be used as a lipid nanoparticle for mRNA-based vaccine formulations.

Compositions provided herein comprise disaggregated spherical nanostructures comprising Quil-A and DOTAP. Compositions of Quil-A and DOTAP as used herein may also be called QTAP, where “Q” refers to Quil-A and “TAP” refers to DOTAP. As used herein, “disaggregated,” refers to the formation of discrete observable particles as opposed to aggregated non-discrete assemblies with non-distinct boundaries. The composition of Quil-A and DOTAP typically includes ratios in a range between about 2:1 to about 1:2 of Quil-A and DOTAP. In some embodiments the final solution of Quil-A is about 0.02% to about 0.001%. In some embodiments the final solution of DOTAP is about 0.1% to about 0.008%. In some embodiments the mRNA, Quil-A, DOTAP, and the buffers may be combined such that the composition has an NP ratio of 4.05 to form the composition described herein. The NP ratio refers to the ratio of positively-chargeable polymer (DOTAP) amine (N=nitrogen) groups to negatively-charged nucleic acid (mRNA) phosphate (P) groups. Thus, an NP ratio of 4.05 infers that 4.05 amine groups are required for binding 1 molecule of mRNA in our formulation. In the absence of any payload molecule, the nanostructure may be between about 50 nm and 100 nm in diameter and is positively charged.

### Payload Molecules

The compositions provided herein may further comprise a payload molecule. The payload molecule may be an antigen of interest for use in a vaccine composition. The payload molecule may be an immunogen for use in a vaccine composition. The payload molecule may be, but is not limited to, a DNA molecule, an RNA molecule, a polynucleotide, a protein, a polypeptide, a virus, a microbe, an attenuated virus, an attenuated microbe, a small molecule, an antibody, or a mixture thereof. In some embodiments, the payload is a virus or bacteria, or a viral or bacterial antigen. In some embodiments, the payload is a live attenuated microbe. In some embodiments, the payload is a recombinant viral vector.

The payload may include a recombinant nucleic acid. Recombinant nucleic acids may encode an immunogenic agent from a pathogen of interest such as, but not limited to, bacterial genes, viral genes and fungal genes. The payload molecule may be a polynucleotide, e.g., a DNA polynucleotide or an RNA polynucleotide, encoding a microbial, e.g., bacterial or viral, antigen. The payload molecule may be an mRNA encoding a bacterial or viral antigen, e.g., an antigen derived from, e.g., Infectious Bronchitis Virus (IBV), Severe Acute Respiratory Syndrome Coronavirus 2 (SARS-CoV-2), influenza virus, Mycobacterium avium subspecies paratuberculosis, Mycobacterium bovis, Mycobacterium tuberculosis, Mycobacterium avium subspecies avium, or Mycobacterium avium subspecies hominissuis. In some embodiments, the payload is a recombinant RNA or DNA molecule encoding an immunogenic or antigenic polypeptide. In some embodiments, the payload is a polynucleotide construct. As used herein, the term “construct” refers to recombinant polynucleotides including, without limitation, DNA and RNA, which may be single-stranded or double-stranded and may represent the sense or the antisense strand. Recombinant polynucleotides are polynucleotides formed by laboratory methods that include polynucleotide sequences derived from at least two different natural sources or they may be synthetic. Constructs thus may include new modifications to endogenous genes introduced by, for example, genome editing technologies. Constructs may also include recombinant polynucleotides created using, for example, recombinant DNA methodologies. A construct may include promoters or enhancers to aid in production of a polypeptide from the polynucleotide.

The payload may include a nucleic acid including RNA or DNA. In some embodiments, the payload includes an RNA molecule. The RNA molecule may be coding RNA, such as messenger RNA (mRNA) or non-coding RNA, such as ribosomal RNA, small nuclear RNA, small nucleolar RNA, piwi-interacting RNA, microRNA, packaging RNA or long noncoding RNA. The RNA may be natural or synthetic including recombinant RNA, guide RNA, single guide RNA, crispr RNA (crRNA) or tracrRNA. The payload may include a DNA molecule. The DNA may be natural or synthetic including, but not limited to plasmid DNA, nuclear DNA, mitochondrial DNA, chloroplast DNA, recombinant DNA, cell-free DNA, relaxed circular DNA or tumor DNA.

The payload molecule may be an antigen specific for bacterial, viral, fungal, or parasitic infections caused by a microbial organism or an antigen derived from a bacterial, viral, fungal or parasitic organism, or a polynucleotide encoding the same, such as Infectious Bronchitis Virus (IBV), Severe Acute Respiratory Syndrome Coronavirus 2 (SARS-CoV-2), Influenza Virus Type A and/or Type B, Mycobacterium avium subspecies paratuberculosis, Mycobacterium bovis, Mycobacterium tuberculosis, Mycobacterium avium subspecies avium or Mycobacterium avium subspecies hominissuis. As used herein, “an antigen derived from an organism” refers to an antigen that comprises at least a portion of a peptide or protein found in the organism, e.g., a subunit vaccine comprising a single protein from a virus, e.g., SARS-CoV-2 spike protein.

The payload may be a polynucleotide or polypeptide antigen derived from Severe Acute Respiratory Syndrome Coronavirus 2 (SARS-CoV-2). SARS-CoV-2 includes the major structural proteins spike(S), envelope (E), membrane (M), and nucleocapsid (N). In some embodiments, the payload is selected from the group consisting of the SARS-CoV-2 S, E, M, and N nucleic acids or proteins and fragments thereof. In some embodiments, the payload is a polynucleotide, e.g., an mRNA or a DNA, encoding the SARS-CoV-2 S, E, M, or N protein or fragments thereof.

### Stability of the Disclosed Compositions

A stable composition is one in which the nanostructure maintains the original size, shape and function and the payload, if present, is not released from the nanostructure. Composition stability may change depending on temperature and time and the presence and type of a payload molecule. In some embodiments, the composition provided herein is as stable as a freshly prepared composition after 72 hrs at −80° C., −20° C., 4° C., 20° C. and 37° C. and any temperatures in-between any of these recited temperatures or as stable as a freshly prepared composition at −80° C., −20° C. or 4° C. and all temperatures in between for 21 days. In some embodiments, the compositions provided herein may be stable for greater than three weeks at any temperature less than −20° C. with a payload molecule. For example, the disclosed compositions may be stable for about 1 day, about 2 days, about 3 days, about 4 days, about 5 days, about 6 days, about 7 days, about 8 days, about 9 days, about 10 days, about 11 days, about 12 days, about 13 days, about 14 days, about 15 days, about 16 days, about 17 days, about 18 days, about 19 days, about 20 days, about 21 days, or more. For example, the disclosed compositions comprising a payload comprising a polynucleotide, e.g., a DNA molecule or an RNA molecule, e.g., an mRNA, may be stable for about 1 day, about 2 days, about 3 days, about 4 days, about 5 days, about 6 days, about 7 days, about 8 days, about 9 days, about 10 days, about 11 days, about 12 days, about 13 days, about 14 days, about 15 days, about 16 days, about 17 days, about 18 days, about 19 days, about 20 days, about 21 days, or more.

### Methods of Forming the Disclosed Compositions

Another aspect of the present disclosure provides methods of forming (or preparing) a composition comprising Quil-A and DOTAP spherical nanostructures are provided. In some embodiments, the methods comprise the steps of: (i) Diluting Quil-A in a buffer, wherein Quil-A is present at a final concentration of about 0.02% to about 0.001%; (ii) diluting DOTAP in a buffer in a separate container from Quil-A, wherein DOTAP is present at a final concentration of about 0.1% to about 0.008%; (iii) combining the contents of (i) with the contents of (ii) and vortexing the combination, e.g., at high speed, for at least 15 seconds and iv) incubating the combined vortexted solution of (iii) whereby a composition comprising Quil-A and DOTAP nanostructures are formed. In some embodiments, the Quil-A is diluted in step (i) to a final concentration of about 0.002% and DOTAP is diluted in step ii to a final concentration of about 0.016%. As used herein, “vortexing,” and grammatical variations thereof, refers to mixing by agitation, e.g., using a vortex mixer.

The buffer of (i) may be or may include sodium sulfate buffer. The buffer of (ii) may be or may include sodium acetate buffer. Other suitable buffers may also be used, including but not limited to piperazine-N, N′-bis (PIPES), 2-ethanesulfonic acid (MES), 3-{[1,3-dihydroxy-2-(hydroxymethyl) propan-2-yl]amino}-2-hydroxypropane-1-sulfonic acid (TAPSO), 3-{[1,3-dihydroxy-2-(hydroxymethyl) propan-2-yl]amino}propane-1-sulfonic acid (TAPS), 2-{[1,3-dihydroxy-2-(hydroxymethyl) propan-2-yl]amino}ethanesulfonic acid (TES), tris(2-hydroxyethyl)amine (TEA), 2-[bis(2-hydroxy)ethylamino] acetic acid (Bicine), 2-[bis(2-hydroxyethyl)amino]-2-(hydroxymethyl) propane-1,3-diol (BTM), 2,2′-(1,3-Propanediyldiimino)bis [2-(hydroxymethyl)-1,3-propanediol] (BTP) or potassium sodium tartrate tetrahydrate (PSTT). Additional buffering agents are sodium phosphate, potassium phosphate, sodium citrate, calcium lactate, sodium succinate, sodium glutamate, sodium bicarbonate, and potassium bicarbonate.

The buffer of step (i) may additionally comprise a payload molecule. The payload molecule may be any payload molecule described herein A composition comprising Quil-A and DOTAP nanostructures is produced by the method described herein and may include a payload molecule. The composition may comprise a vaccine formulation. The vaccine formulation may comprise a payload molecule as described herein. The compositions provided herein may comprise a vaccine formulation and may further include an adjuvant. An adjuvant is a substance that increases or modulates the immune response to a vaccine. In some embodiments the vaccine formulation comprises other suitable ingredients or agents. The vaccine formulation may include an antigen and/or a pharmaceutically acceptable carrier. As used herein, the term “carrier” refers to a pharmaceutically acceptable solid or liquid filler, diluent or encapsulating material. A water-containing liquid carrier can contain pharmaceutically acceptable additives such as acidifying agents, alkalizing agents, antimicrobial preservatives, antioxidants, buffering agents, chelating agents, complexing agents, solubilizing agents, humectants, solvents, suspending and/or viscosity-increasing agents, tonicity agents, wetting agents or other biocompatible materials. A tabulation of ingredients listed by the above categories, may be found in the U.S. Pharmacopeia National Formulary, 1857-1859, (1990).

Some examples of the materials which can serve as pharmaceutically acceptable carriers are sugars, such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil, safflower oil, sesame oil, olive oil, corn oil and soybean oil; glycols, such as propylene glycol; polyols such as glycerin, sorbitol, mannitol and polyethylene glycol; esters such as ethyl oleate and ethyl laurate; agar; buffering agents such as magnesium hydroxide and aluminum hydroxide; alginic acid; pyrogen free water; isotonic saline; Ringer's solution, ethyl alcohol and phosphate buffer solutions, as well as other nontoxic compatible substances used in pharmaceutical formulations. Wetting agents, emulsifiers and lubricants such as sodium lauryl sulfate and magnesium stearate, as well as coloring agents, release agents, coating agents, sweetening, flavoring and perfuming agents, preservatives and antioxidants can also be present in the compositions, according to the desires of the formulator.

Examples of pharmaceutically acceptable antioxidants include water soluble antioxidants such as ascorbic acid, cysteine hydrochloride, sodium bisulfite, sodium metabisulfite, sodium sulfite and the like; oil-soluble antioxidants such as ascorbyl palmitate, butylated hydroxyanisole (BHA), butylated hydroxytoluene (BHT), lecithin, propyl gallate, alpha-tocopherol and the like; and metal-chelating agents such as citric acid, ethylenediamine tetraacetic acid (EDTA), sorbitol, tartaric acid, phosphoric acid and the like.

The present compositions and formulations may also comprise other suitable agents such as a stabilizing delivery vehicle, carrier, support or complex-forming species. The coordinate administration methods and combinatorial formulations of the instant invention may optionally incorporate effective carriers, processing agents, or delivery vehicles, to provide improved formulations for delivery of the QAC complex and other biologically active agents and antigens of the composition. Other excipients, if desired, may be included as part of the final vaccine formulation.

### Methods of Generating an Immune Response

Another aspect of the present disclosure provides a method of generating (or eliciting) an immune response in a subject are provided. In some embodiments, the methods comprise the step of administering to a subject a composition described herein or the vaccine compositions described herein. The term “subject” may be used interchangeably with the terms “individual” and “patient” and includes human and non-human mammalian subjects. As used herein, a “subject in need thereof” refers to a subject in need of protection from a disease condition, e.g., a subject in need of elicitation of an immune response to a particular antigen or a subject in need of vaccination against infection by a particular organism or microbe, or treatment of an indicated disease or disorder.

As used herein, the term “administering” an agent, such as the compositions described herein to an animal or cell, is intended to refer to dispensing, delivering or applying the substance to the intended target. In terms of the composition, the term “administering” is intended to refer to contacting or dispensing, delivering or applying the therapeutic agent to a subject by any suitable route for delivery of the therapeutic agent to the desired location in the animal, including delivery by either the parenteral or oral route, e.g., intramuscular injection, subcutaneous/intradermal injection, intravenous injection, intrathecal administration, buccal administration, transdermal delivery, topical administration, in ovo administration and administration by the intranasal or respiratory tract route.

Generating, eliciting, or inducing an immune response refers to the activation of any component of the immune system in a subject subsequent to treatment with a particular antigen. In some embodiments, generating an immune response includes generating a humoral immune response. Humoral immunity refers to antigen-specific antibodies which are produced by B cells. Humoral immune responses comprise the production of antibodies, complement proteins, and antimicrobial peptides. An immune response generated by the methods and compositions provided herein may further comprise the production of cytokines including but not limited to production of one or more of interferon gamma (IFNγ), interleukin 1 beta (IL-1B), tumor necrosis factor alpha (TNF-α), interleukin 2 (IL-2), interleukin 6 (IL-6), interleukin 21 (IL-21), interleukin 4 (IL-4), interleukin 5 (IL-5), and interleukin 9 (IL-9). Methods of detecting the foregoing molecules are known in the art including, e.g., enzyme linked immunosorbent assays (ELISAs) directed to the molecules. An immune response generated by the methods and compositions provided herein may further comprise the production or activation of other immune cells including, but not limited to macrophages, dendritic cells, B lymphocytes, T lymphocytes, polyfunctional T lymphocytes, neutrophils, monocytes, and plasma cells. Methods of determining the activation of the foregoing immune cell types are known in the art and may comprise, e.g., the use of flow cytometry for the detection of well-established markers of activation, e.g., CD45RO for activated human T cells, or CD69 for activated mouse T cells. Methods of determining antigen-specific elicitation of an immune response are known in the art and include, e.g., ELISA assays or MHC-tetramer analysis. In some embodiments, the composition comprises a payload, wherein the payload is any payload molecule described herein. In some embodiments the payload molecule can be detected up to about 30 days, including about 29, about 28, about 27, about 26, about 25, about 24, about 23, about 22, about 21, about 20, about 19, about 18, about 17, about 16, about 15, about 14, about 13, about 12, about 11, about 10, about 9, about 8, about 7, about 6, about 5, about 4, about 3, about 2, or about 1 day after administration of the composition.

### Methods of Delivering a Payload Inside a Cell

Another aspect of the present disclosure provides a method of delivering a payload inside a cell (also referred to as methods of introducing a payload into a cell or methods of transfecting a a cell) comprising contacting the cell with the compositions provided herein. For example, In some embodiments the cell is contacted by the composition in vitro. In some embodiments the cell is contact in vivo, or ex vivo. The composition contacting the cell may be any composition provided herein and may comprise a payload molecule. In some embodiments, the payload molecule is a polynucleotide or a nucleoprotein complex (a nuclease such as a Cas9 and an associated guide RNA).

As used herein, the terms “polynucleotide,” “polynucleotide sequence,” “nucleic acid” and “nucleic acid sequence” refer to a nucleotide, oligonucleotide, polynucleotide (which terms may be used interchangeably), or any fragment thereof. These phrases also refer to DNA or RNA of natural or synthetic origin (which may be single-stranded or double-stranded and may represent the sense or the antisense strand). The polynucleotides may comprise or consist of cDNA or genomic DNA. Polynucleotides homologous to the polynucleotides described herein are also provided. Those of skill in the art understand the degeneracy of the genetic code and that a variety of polynucleotides can encode the same polypeptide. A nucleoprotein comprises a protein linked to a nucleic acid. In some embodiments, the nucleic acid is a gRNA and the nucleoprotein comprises a CRISPR associated protein (Cas) complexed with a gRNA. In some embodiments the payload molecule is a DNA polynucleotide, including plasmid DNA.

### Miscellaneous

Unless otherwise specified or indicated by context, the terms “a”, “an”, and “the” mean “one or more.” For example, “a molecule” should be interpreted to mean “one or more molecules.” As used herein, “about”, “approximately,” “substantially,” and “significantly” will be understood by persons of ordinary skill in the art and will vary to some extent on the context in which they are used. If there are uses of the term which are not clear to persons of ordinary skill in the art given the context in which it is used, “about” and “approximately” will mean plus or minus≤10% of the particular term and “substantially” and “significantly” will mean plus or minus >10% of the particular term.

As used herein, the terms “include” and “including” have the same meaning as the terms “comprise” and “comprising.” The terms “comprise” and “comprising” should be interpreted as being “open” transitional terms that permit the inclusion of additional components further to those components recited in the claims. The terms “consist” and “consisting of” should be interpreted as being “closed” transitional terms that do not permit the inclusion of additional components other than the components recited in the claims. The term “consisting essentially of” should be interpreted to be partially closed and allowing the inclusion only of additional components that do not fundamentally alter the nature of the claimed subject matter.

All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

All references, including publications, patent applications, and patents, cited herein are hereby incorporated by reference to the same extent as if each reference were individually and specifically indicated to be incorporated by reference and were set forth in its entirety herein.

Preferred aspects of this invention are described herein, including the best mode known to the inventors for carrying out the invention. Variations of those preferred aspects may become apparent to those of ordinary skill in the art upon reading the foregoing description. The inventors expect a person having ordinary skill in the art to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

### EXAMPLES

The following Examples are illustrative and should not be interpreted to limit the scope of the claimed subject matter.

### Example 1-Protective RNA Nanovaccines Against Mycobacterium avium Subspecies Hominissuis

Reference is made to the manuscript: Touray et al., “Protective RNA Nanovaccines Against Mycobacterium avium subspecies hominissuis,” the content of which is incorporated herein by reference in its entirety.

### 1 Introduction

Nucleic acid (NA) vaccines have demonstrated improved safety and tolerability compared to traditional vaccines (1-4), especially with the worldwide use of an mRNA vaccine to control the SARS-CoV-2 pandemic. Unlike live-attenuated vaccines (LAV), NA vaccines encode individual or a myriad of immunogenic antigens to trigger protection without the untoward effect of other factors encoded by the LAV. However, NA vaccines are often less immunogenic than LAV and therefore need to be formulated with an adjuvant to boost their immunogenicity (5-7). Over the past few decades, progress has been made toward identifying novel adjuvants to boost the immune response generated by NA vaccines against both infectious and non-infectious diseases (5, 8-11) including a class of lipid nanoparticles (LNPs) to help with the delivery of vaccine antigens. LNPs have been used as both adjuvants and delivery vehicles for mRNA vaccine constructs (12-14). The LNPs protect mRNA from host endonucleases and promote efficient cellular uptake of constructs for efficient protein translation in cells (15-17). LNPs made from cationic lipids such as 1,2-di-O-octadecenyl-3-trimethylammonium-propane (DOTMA) and its biodegradable analog DOTAP are part of mRNA-based vaccine formulations against several cancers and autoimmune encephalomyelitis (18, 19). Recently, LNP adjuvants were used in two mRNA-based vaccines created by BioNTech/Pfizer and Moderna that targets SARS-Cov-2 virus and have shown protective efficacy in both animal and human studies (20, 21). In this report, we developed a nanoadjuvant system of both Quil-A and DOTAP called QTAP. The developed QTAP nanovaccine adjuvant was tested against M. ah causing pulmonary infection in immunocompromised patients.

Adjuvant systems are a combination of immune stimulants that enhance the immunogenicity of vaccine antigens. A purified version of Quil-A (QS-21) has been shown to be less toxic in both mice and humans and is part of approved vaccines against malaria (Mosquirix) and shingles (Shingrix) with high immunogenicity and protective efficacy (22, 23). The QS-21 stimulates both antibody-based and cell-mediated immune responses, eliciting a Th-1-biased immune response with the production of high titers of antibodies (IgG2a and IgG2b, in addition to IgG1), as well as antigen-specific cytotoxic T lymphocytes (24). These studies clearly demonstrate the importance of adjuvants in vaccine formulations especially against challenging pathogens. Combining the efficacy of mRNA vaccines, delivery functions of LNPs, and the inflammatory effect of adjuvants may be a suitable approach to enhance the overall efficacy of nucleic acid-based vaccines.

The application of mRNA vaccines against intracellular pathogens for which no effective vaccine has been developed remains unexplored (25). Currently, there is no licensed vaccine for M. ah infection, a significant health problem for the aging and immunocompromised population (26, 27). In pursuit of addressing this problem, several platform technologies were tested before including nucleic acid (NA) vaccines (28-30). Mycobacteria rely on a plethora of antigens to drive its virulence in the host making vaccine development a challenge (31, 32), and M. ah is not an exception. Previously, RNA as a booster to protein vaccines against M. ah showed protective efficacy in mice (33). We suggest that mRNA vaccines targeting a complex pathogen such as M. ah would require a mixture of antigens combined with an efficient adjuvant system to provide protective immunity, as suggested before (33). In this report, we describe our efforts towards the development of nanoadjuvant systems with improved physicochemical characteristics and their application to generate protective immunity against aerosol challenges with M. ah. To the best of our knowledge, this is the first nanoadjuvant system that combines DOTAP, Quil-A (QTAP), and mRNA at a ratio that ensures efficient cellular uptake, mRNA delivery, protein translation, enhanced immune activation and elicits protective immunity in a murine challenge model. Not only QTAP-based constructs were more stable for prolonged times at different storage temperatures, but also, they were able to efficiently transfect immune cells and induce their activation without antigens. Moreover, when antigens were added, the QTAP adjuvanted mRNA (QTAP-Ag85B+Hsp70) increased macrophage activation and generated localized immunity in immunized mice with the presence of polyfunctional T-cells and reduced tissue colonization with the challenge strain of M. ah. Together, these results provide clear evidence of the novel QTAP as a transfection and vaccine nanoadjuvant system that can efficiently deliver mRNA constructs and elicit a protective immune response against M. ah infection and potentially other intracellular pathogens.

### 2 Materials and Methods

### 2.1 Bacterial Cultures and Plasmids

For all challenge studies, a Mycobacterium avium subspecies hominissuis clinical isolate from the collection of the Wisconsin State Laboratory of Hygiene (designated M. ah W14 or M. ah) was grown and its genome sequenced as detailed before (34). For culturing, M. ah W14 was grown in Middlebrook 7H9 broth (BD Biosciences, Sparks, MD, USA) supplemented with 10% DC (2% glucose, 5%, and 0.85% NaCl) in a shaking incubator at 37° C. Bacterial cultures were harvested and stored as before (35). Sequences for mycobacterial genes (hsp70, Ag85B), green fluorescent protein (GFP) from jellyfish Aequorea Victoria, and Luciferase (Luc) gene from firefly luciferase were downloaded from GenBank and amplified followed by cloning onto an expression vector pCMV from our laboratory collection, as described before (36). These vectors were used as templates for in vitro RNA synthesis using HiScribe® T7 ARCA mRNA Kit (with tailing) (NEB, Ipswich, MA, USA).

### 2.2 Animal Vaccinations

To examine nanovaccine safety and immunogenicity, preparations of QTAP-mRNA encoding mycobacterial antigens (Ag85B and Hsp70) were evaluated in 3-weeks old C57BL/6 mice. Mice were purchased from Jackson Laboratory (Bar Harbor, ME, USA) and randomly divided into 3 groups (N=14/group), inoculated through the subcutaneous route with 3 doses (15 μg each) of QTAP-mRNA encoding different antigens (Ag85B+Hsp70) at 5-weeks intervals while other groups were inoculated with PBS or 15 μg of QTAP alone to serve as controls. Mice were monitored for general distress, depression, or inappetence and weight changes over the course of the 15 weeks. At each vaccination time-point, blood samples were collected, and serum was separated for cytokine analysis. For some groups used to examine vaccine protective immunity, mice (N=3) were euthanized from each group at 5 weeks after final immunization to harvest lung and spleen for flow cytometry and histopathology. The remaining 12 mice in each group were infected with 100 CFUs of M. ah through the aerosol route. After 48 h, mice (N=2) were euthanized from each group, and their lungs and spleen were harvested and homogenized for infection dose determination by CFU count. At 4 and 8 weeks post-challenge, mice (N=5) were euthanized from each group and their organs (lung, spleen, liver) were harvested and homogenized for flow cytometry, histopathology, and CFU enumeration as detailed before (36, 37).

### 2.3 Preparation and Characterization of QTAP Nanovaccines

Modified mRNA was synthesized using the HiScribe™ T7 ARCA mRNA Kit (with tailing) (New England BioLabs (NEB) E2060S) and Pseudouridine-5′-Triphosphate-(TriLink N-1019) (San Diego, CA, USA). Briefly, capped modified mRNAs were synthesized by co-transcriptional incorporation of Anti-Reverse Cap Analog (ARCA, NEB #S1411) using T7 RNA Polymerase in the presence of 10 mM Pseudo-UTP. This is followed by DNase I treatment to remove template DNA, and treatment with poly (A) polymerase for poly (A) tail addition. The resulting mRNA is purified by column purification, quantified by Nanodrop, and quality assessed using gel electrophoresis.

To prepare QTAP-mRNA, DOTAP (18:1 TAP (DOTAP) 890890) was purchased from Avanti Polar Lipids (Birmingham, AL, USA) without purification and dissolved in 2% glucose water to a final concentration of 10%. The Quil-A (VET-SAP, Desert King) stock solution of 0.2% was made in nuclease-free water. For each preparation, mRNA, Quil-A, DOTAP, and the buffers were combined at a nitrogen to phosphate (NP) ratio of 4.05 to form QTAP-mRNA. Size distribution and zeta potential of QTAP-mRNA in aqueous dispersion were measured by dynamic light scattering (DLS) on a Malvern Zetasizer instrument at 25° C. For zeta potential measurement, an aliquot (5 μl) of QTAP-NPs was diluted in Alpha-q water and placed in a disposable capillary zeta potential cell, available from the Zetasizer Nano series (38). Transmission Electron Microscopy was performed at the Medical School Electron Microscopy Facility of the University of Wisconsin-Madison using a Philips CM120 transmission electron microscope (FEI, Eindhoven, the Netherlands) at 80 kV. For encapsulation efficiency QTAP-NPs loaded with mRNA were resuspended in 600 μl of 0.05 M phosphate-buffered saline (PBS, pH 7.4) at 37° C. At each time point, suspensions were removed and centrifuged at 14,000 relative centrifugal force for 10 min. The supernatant was removed and replaced with PBS and returned to incubation. Supernatant samples were quantified for released mRNA from the QTAP using a NanoDrop spectrophotometer and compared to the total mRNA used (39, 40).

### 2.4 Cell Viability and Transfection Efficiency of QTAP

Cell viability following mRNA transfection was measured using MTT assay (Millipore Sigma 11465007001, Burlington, MA, USA). Baby Hamster Kidney (BHK) cells (American Type Culture Collection (ATCC), Manassas, VA) were cultured in 96-well plates and transfected for 24 h at 37° C. and 5% CO2. The medium was removed and replaced with 10 μl of the MTT labeling reagent (final concentration 0.5 mg/ml) and incubated for 4 h at 37° C. and 5% CO2. A 100 μl of the solubilization solution was added to each well and incubated at 37° C. and 5% CO2 overnight. The absorbance was recorded using an ELISA plate reader at wavelength 550. To determine transfection efficiency, BHK cells, HEK293T cells, were cultured in Dulbecco's modified Eagle's medium (DMEM) (Gibco 31966-021, Waltham, MA, USA) medium supplemented with 10% FBS (Sigma F7524) and penicillin-streptomycin (Gibco 15140-122) whiles J774. A macrophages were cultured in RPMI 1640 (Corning 10-040-CM) medium supplemented with 10% FBS (Sigma F7524) and penicillin-streptomycin (Gibco 15140-122). Cells were seeded at 300,000 density and incubated at 37° C. and 5% CO2 until they reach 70-80% confluency followed by transfection with QTAP-NPs encapsulating mRNA. A commercial transfection reagent TransIT®-mRNA Transfection Kit (Mirus 2250, Madison, WI) and in-house made DOTAP-NPs were used to transfect an equal amount of mRNA in separate wells as transfection controls. At 24 h, 48 h, and 72 h post-transfection, media were removed, and cells were washed with PBS. Cells were lifted from the plate by gently pipetting up and down and transferred to a 2 ml centrifuge tube and centrifuged at 1500 g for 5 minutes at 4° C. For flow cytometry, cells were run on a BD LSR Fortessa flow cytometer. Data were analyzed with FlowJo software (BD Bioscience). The strategy for gating on GFP+ cells was debris exclusion on the forward scatter (FSC) vs side scatter (SSC) dot plot, followed by exclusion of dead cells by fixability dye eFluor 780 (number 65-0865-14; Invitrogen) staining. From the live cell population, total GFP+ cells were gated. Finally, the mean fluorescence intensity of the GFP+ population was determined.

### 2.5 Western Blot Analysis

Cells were transfected with mRNA as described above. After 24 h of incubation, cells were detached and washed with ice-cold PBS, lysed with 1% (w/v) SDS, followed by sonication using Misonix Ultrasonic Liquid Processor sonicator 3000. The total protein for each sample was separated by SDS-PAGE, and transferred to a poly (vinylidene difluoride) membrane; proteins were detected by western analysis with the histidine-tag antibodies ad detailed before (41).

### 2.6 Flow Cytometric Assessment of QTAP-mRNA Immunogenicity and Protective Efficacy

Lungs and spleens collected from vaccinated mice were used for flow cytometric assessment. Briefly, tissues were excised and placed in a gentleMACS dissociator M Tube (Miltenyi 130-093-236, Bergisch Gladbach, Germany) with 3 mL collagenase B (1 mg/mL) (Roche, Basel, Switzerland) and incubated for 30 min at 37° C. Single-cell were prepared by gently squeezing through a 70-mm cell strainer (Falcon) after lysing RBCs using 1×BD Biosciences BD Pharm Lyse™ (San Jose, CA, USA). For intracellular cytokine staining, 10⁶ cells were stimulated with M. ah purified protein derivative (PPD) (1 mg/ml) and IL-2 (400 U/ml) while an equal concentration of IL-2 was added to the remaining replicate as unstimulated control. After 16 h incubation at 37° C., 5% CO₂, Brefeldin A (1 uL/mL, GolgiPlug, BD Biosciences) was added, and the cells were further incubated for an additional 6 h at 37° C., 37° C., 5% CO₂. Fluorochrome-labeled antibodies against the cell-surface antigens CD4 (BUV 496, GK1.5), CD8a (BUV395, 53-6.7), and intracellular antigens IFN-γ (APC, XMG1.2); TNF-α (BV421, MP6-XT22); IL-2 (PE-CF594, JES6-5H4); IL-17 (FITC, TC11-18H10.1) were purchased from BD Biosciences; Biolegend (San Diego, CA, USA); eBioscience (San Diego, CA, USA); or Invitrogen (Grand Island, NY, USA). Before antibody staining, the cells were stained for viability with Dye eFluor 780 (eBiosciences, San Diego, CA, USA). After stimulation, the cells were stained for surface markers and then processed with the Cytofix/Cytoperm kit (BD Biosciences, San Jose, CA, USA). To stain for cytokines, the cells were first stained for cell-surface molecules, fixed, permeabilized, and subsequently stained for the cytokines. All samples were acquired on an LSR Fortessa flow cytometer (BD Biosciences, San Jose, CA, USA). Data were analyzed with FlowJo software (TreeStar, Woodburn, OR, USA). Results are expressed as the difference in the percentage of stimulated cells with that of unstimulated cells. At least 100,000 events were collected for each sample. A Boolean gating strategy was applied for the determination of cytokine-secreting T cells.

### 2.7 ELISA Assay

Serum samples were collected from animals at designated times and stored at −80° C. until use. After thawing, sera were 1:10 diluted with buffer (PBS-Tween 0.05% with 1% BSA) to obtain a working concentration for the ELISA. ELISA plates (96-well) were coated with M. avium purified protein derivatives (PPD) at a concentration of 10 μg/mL in carbonate-bicarbonate buffer (pH 9.6). The plates were incubated overnight at 4° C., washed with PBS-Tween 0.05%, and blocked with 200 μL of blocking buffer (PBS-Tween 0.05% with 3% BSA) for 1 hour at room temperature. After blocking, plates were washed and 100 μL of the diluted serum samples were added to each well for 2 hours at room temperature. After incubation, the plates were washed and 100 μL of HRP-conjugated anti-mouse IgG antibody (diluted 1:5000 in sample diluent buffer) was added to each well. The plates were then incubated for 1 hour at room temperature, washed and 100 μL of TMB substrate solution was added to each well. After 10-15 minutes of incubation at room temperature, protected from light, color development was stopped by adding 50 μL of 2M sulfuric acid to each well. Plates were read at 450 nm using a microplate reader.

### 2.8 Statistical Analysis

Statistical analyses were performed using GraphPad software (La Jolla, CA, USA). Nanoparticle size, protein expression, thermostability, and cytotoxicity were compared using ordinary one-way ANOVA where *p<0.05, **p<0.01, ***p<0.001, and ****p<0.0001 were considered significantly different among groups. Serum cytokine and cellular immune assays were compared using an ordinary one-way ANOVA test where *, p<0.05; **, p<0.01 were considered significantly different among groups.

### 3 Results

### 3.1 Characterization of QTAP-mRNA Nanovaccine

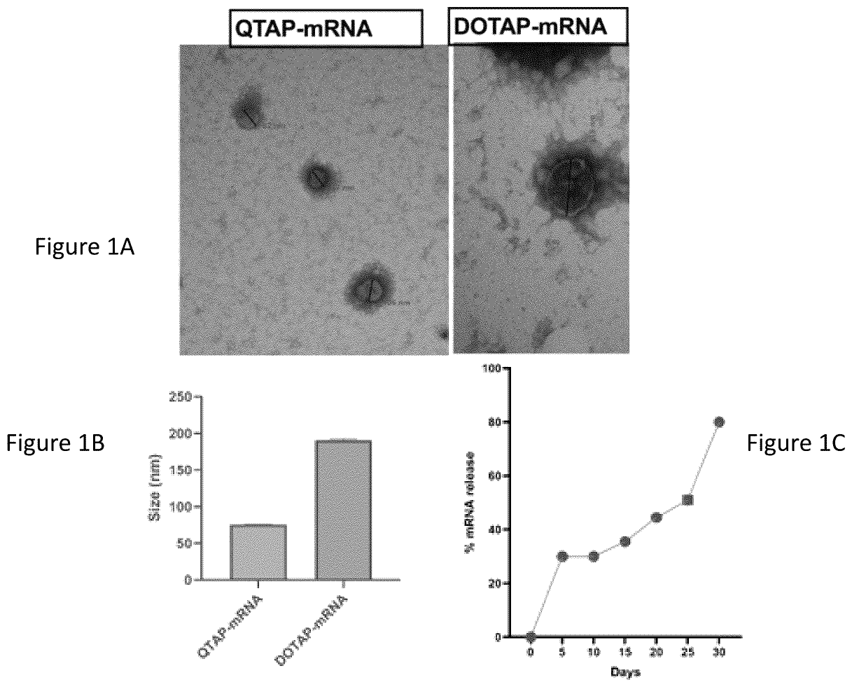

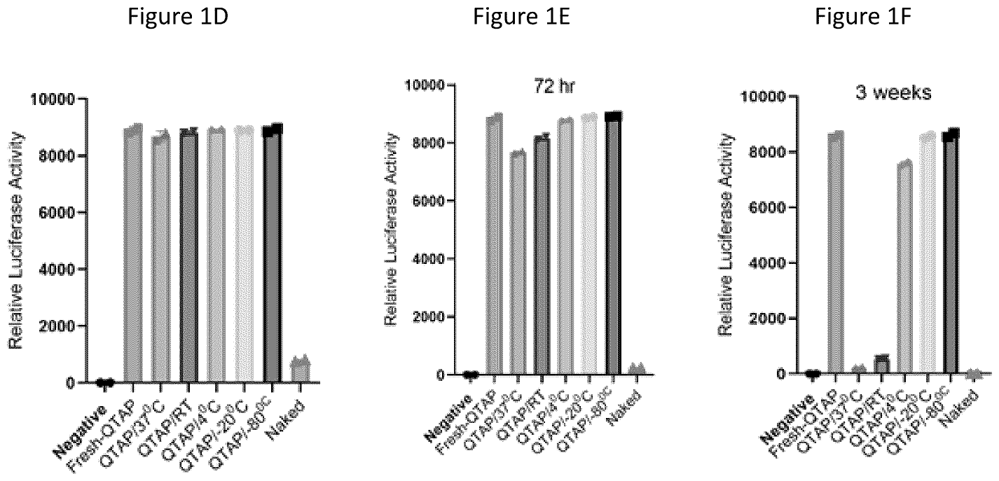

To facilitate the characterization of QTAP-mRNA nanovaccines, we first formulated modified mRNA-encoding reporter genes such as luciferase (Luc) or GFP proteins using QTAP encapsulation. The mRNA was modified with the substitution of at least 66% of the native uridine nucleotides to Pseudouridines (Ψ) as suggested before (42, 43). The Ψ-mRNA integrity and purity were assessed by gel electrophoresis and formulated into the Quil-A adjuvanted DOTAP LNPs (QTAP). Transmission electron microscopy (TEM) analysis of QTAP-mRNA encoding GFP showed a few particles (˜95 nm in size) which are spherical with no observed particle aggregation (FIG. 1A). Also, dynamic light scattering (DLS) of the QTAP-mRNA complex displayed an average particle size of ˜75 nm with a zeta potential of 34 (FIG. 1B). The encapsulation efficiency (EE %) of QTAP nanoparticles (NP) was >90%. The release kinetics of mRNA from QTAP NPs showed sustained release of up to 80% of the mRNA cargo within the first 30 days of testing (FIG. 1C).

To test the stability of QTAP, we encapsulated Y′-mRNA encoding Luc protein in QTAP and incubated the complex at different temperatures. At all temperatures, QTAP-mRNA is stable for up to 72 h with no significant difference to freshly prepared QTAP-mRNA (FIGS. 1D, E). Interestingly, after 3 weeks, QTAP-mRNA remains stable at 4° C., −20° C., and −80° C. whereas at 37° C. and RT, the stability was reduced significantly after 72 h (FIG. 1F). The luciferase activity suggests that QTAP-mRNA forms nanostructures that are stable at higher temperatures and can deliver the mRNA cargo in a sustained release manner.

3.2 the Combination of DOTAP and Quil-A Enhances the Delivery of Functional Modified mRNA in Cells

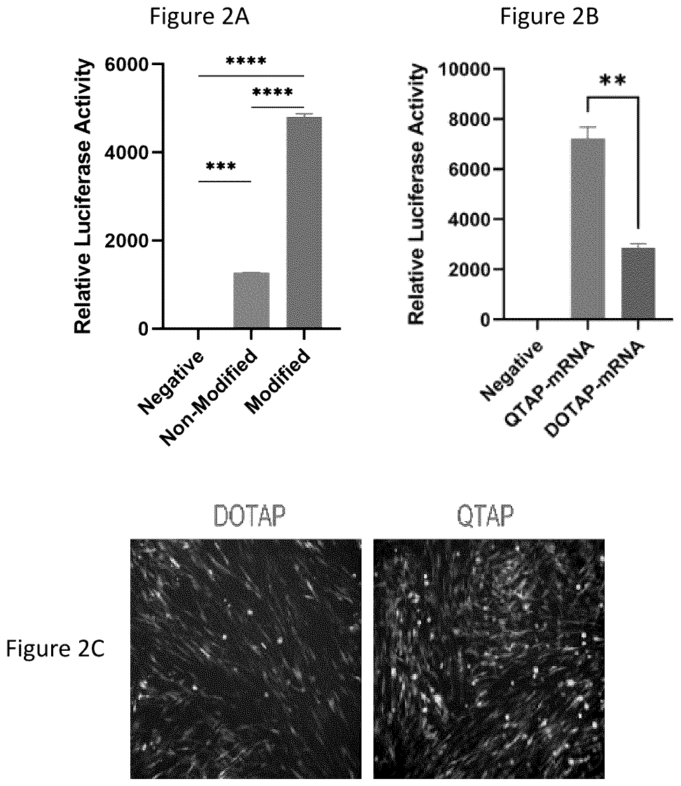

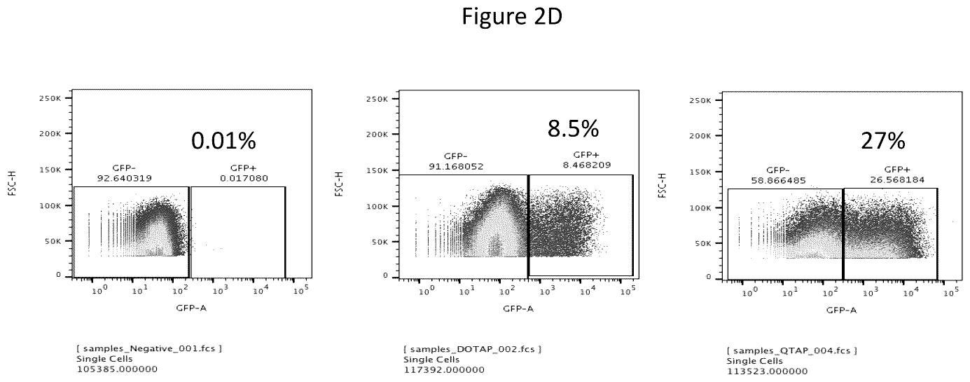

To determine the ability of QTAP to efficiently deliver functional Ψ-mRNA, BHK cells were transfected with Ψ-mRNA encoding either Luc or GFP proteins. At 24 h post-transfection, Ψ modified mRNA resulted in a 4-fold increase in luciferase expression (FIG. 2A). Similarly, luciferase activity was higher in QTAP-mRNA compared to DOTAP-mRNA transfected cells (FIG. 2B). Cells transfected with QTAP encapsulating Y-mRNA encoding GFP showed a higher number of cells expressing GFP than unmodified constructs at 48 h (data not shown) suggesting that enhanced protein expression is not dependent on QTAP encapsulation but Y presence. Moreover, complexation of Y-mRNA encoding GFP with the neoadjuvant (QTAP) showed a significant increase in the number of GFP-expressing cells compared to DOTAP control at 72 h (FIG. 2C). When flow cytometry was used, the number of cells expressing GFP was higher in QTAP-mRNA transfected cells than in DOTAP-mRNA transfected cells (FIG. 2D). Overall, these findings illustrate the ability of the novel QTAP delivery platform to transfect mRNA efficiently in cells leading to detectable protein expression in vitro.

3.3 QTAP NPs Encapsulating Modified mRNA of Mycobacterial Ag85B Activate Macrophages and are not Cytotoxic

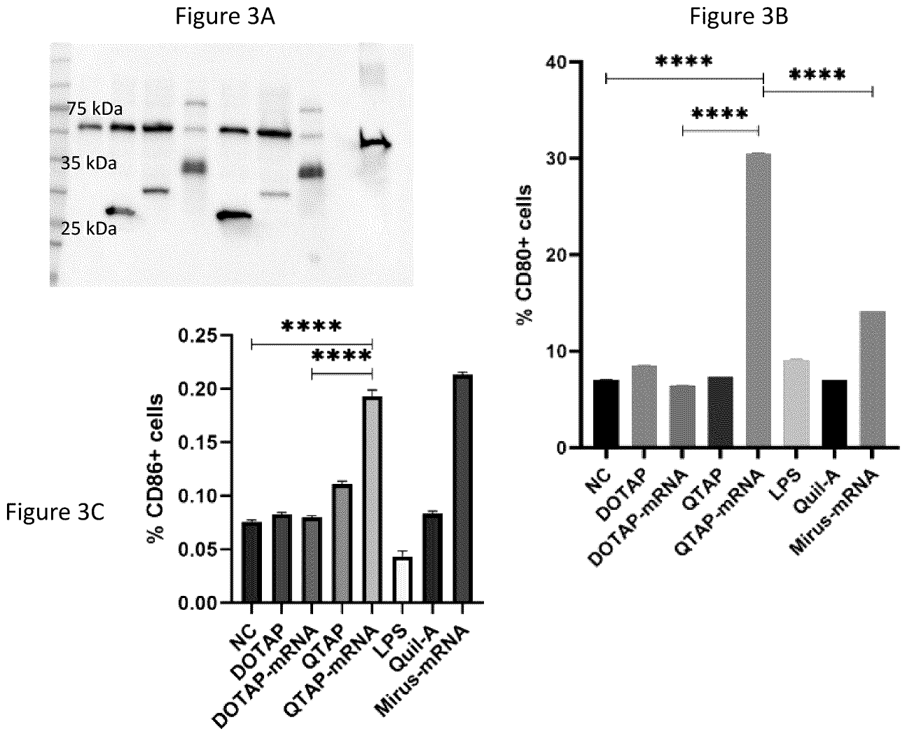

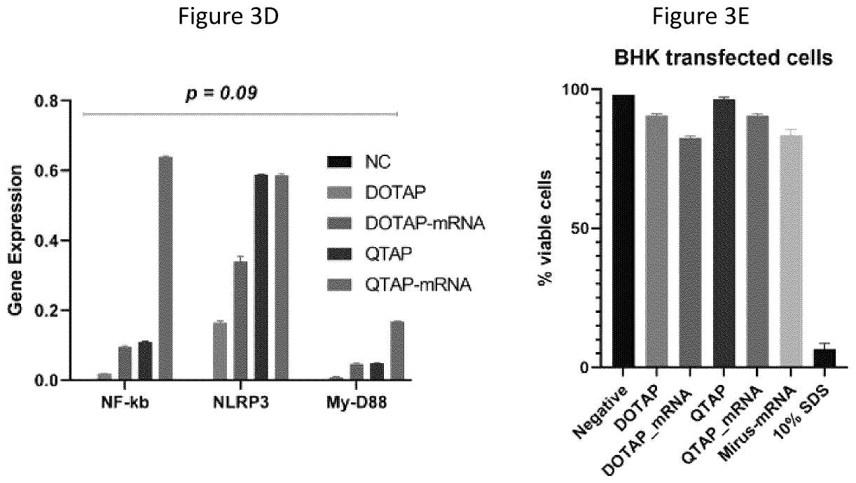

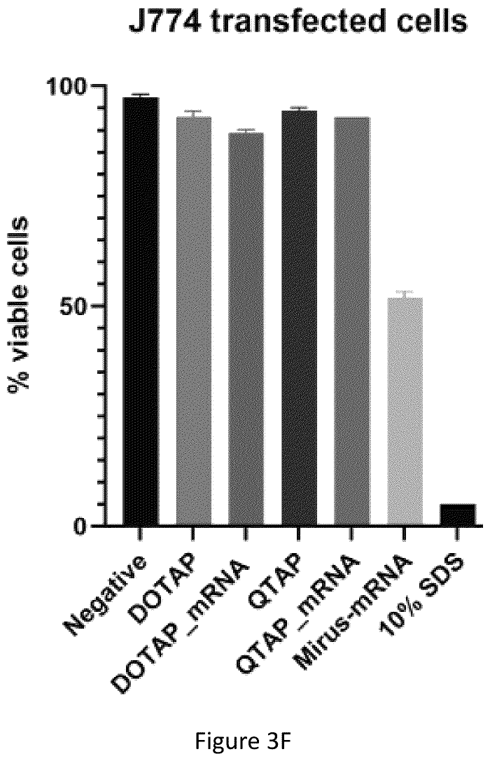

To examine how a QTAP nanovaccine can modulate cells, macrophages (J744A.1) were transfected with QTAP followed by flow cytometric acquisition. Western blot analysis of M. ah antigens Ag85B and Hsp70 showed increased protein expression from BHK cells after transfection with QTAP-mRNA (FIG. 3A). Cells expressing CD80 (FIG. 3B) and CD86 (FIG. 3C) were significantly higher when transfected with QTAP-Y-mRNA-Ag85B than DOTAP-Ψ-mRNA-Ag85B. Interestingly, transfection of cells with either QTAP alone or QTAP-Ψ-mRNA-Ag85B resulted in the upregulation of inflammatory pathways in cell culture (FIG. 3D) compared to DOTAP controls. Overall, the novel QTAP NAS can activate macrophages toward an inflammatory state. Finally, we also analyzed the cytotoxicity of QTAP-Ψ-mRNA using MTT cytotoxicity assay on two different cell lines (BHK and J774). The viability of both BHK and J774 cells transfected with DOTAP and QTAP encapsulating Ag85B Y-mRNA was not significantly affected compared to the negative control (FIGS. 3E, F). On the other hand, Mirus mRNA transfection reagent significantly reduced cell viability in both cells at the same time point. Although at this time point we observed higher transfection efficiency in cells transfected with Mirus mRNA transfection reagent encapsulating Y′-mRNA encoding GFP, it also resulted in lower viability in both cells. Additionally, evaluation of reactive oxygen species (ROS) production in macrophages showed QTAP-mRNA ROS levels comparable to DMSO. However, DOTAP-mRNA has higher ROS levels than both DMSO and QTAP-mRNA (data not shown).

3.4 QTAP-Based mRNA Nanovaccine is Safe and Elicits a Robust Immune Response in Mice.

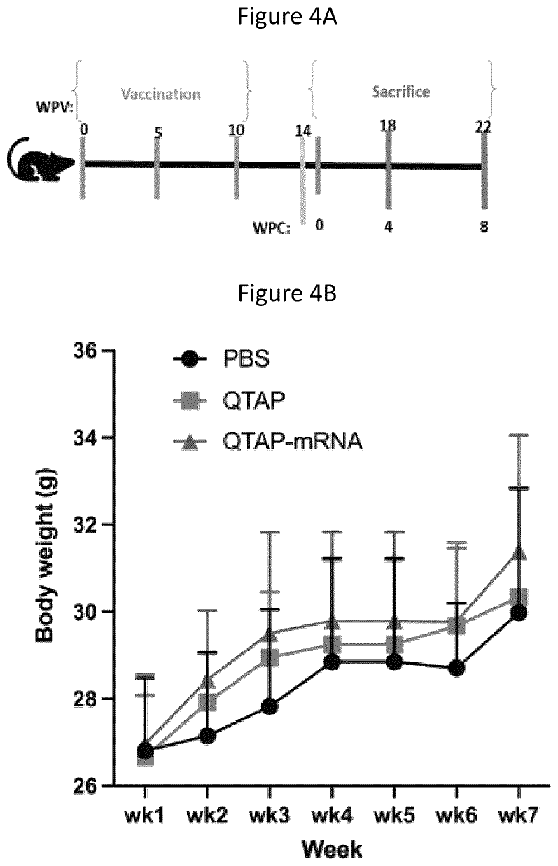

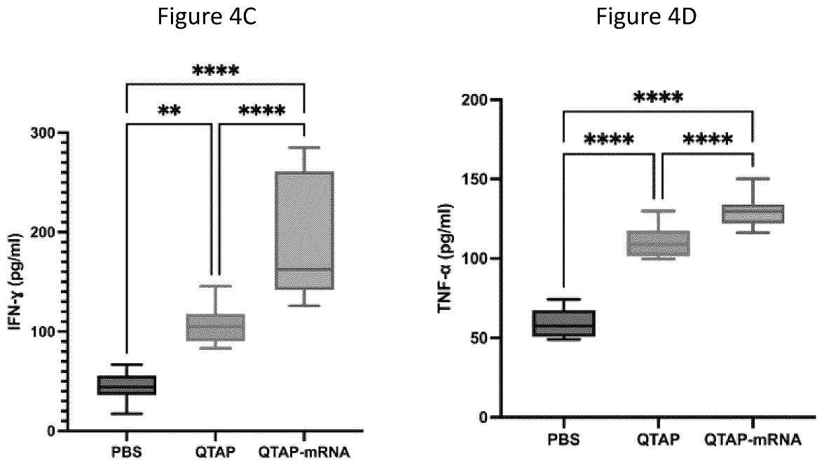

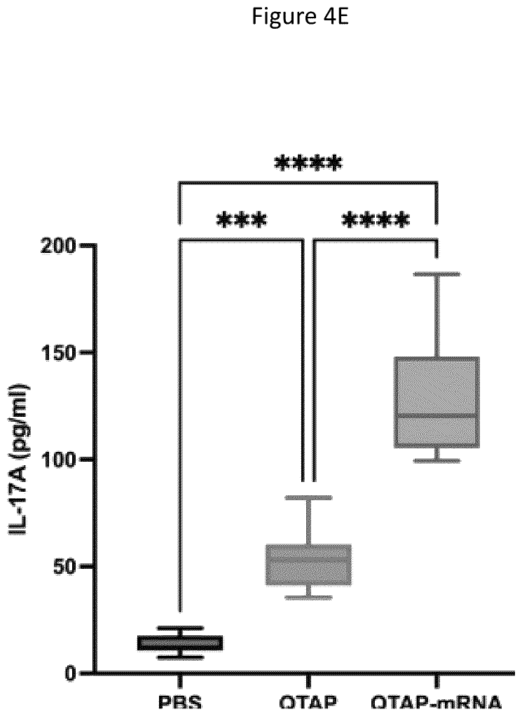

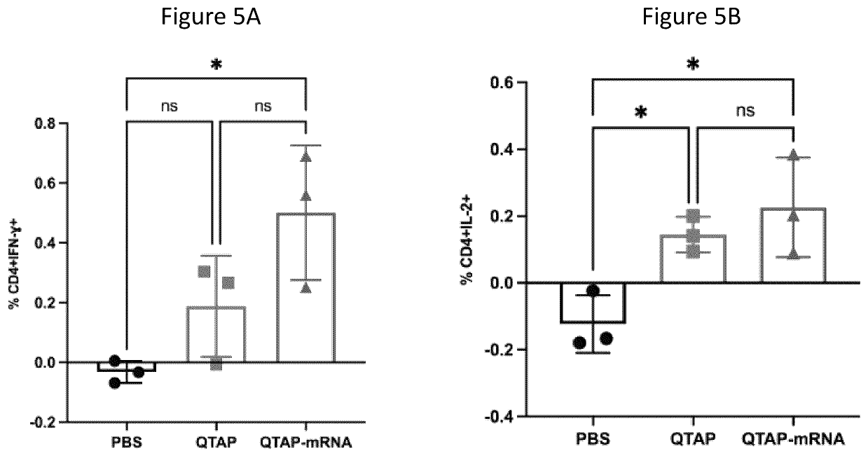

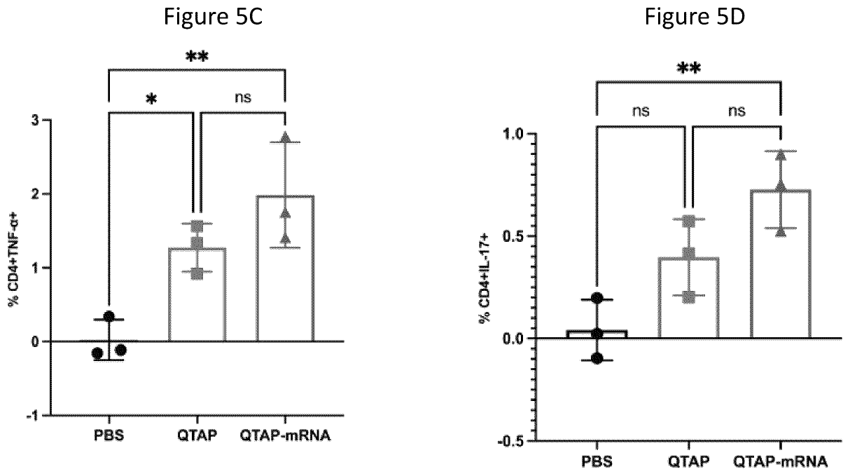

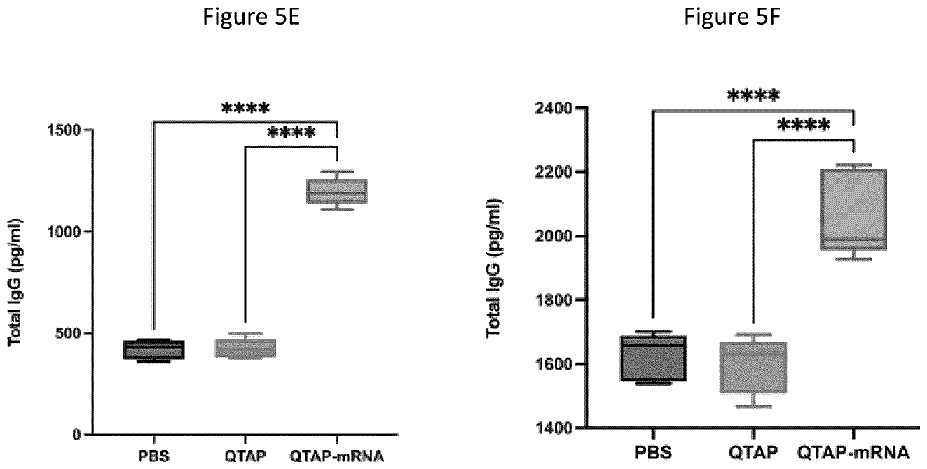

The safety and immunogenicity of QTAP encapsulating modified mRNA encoding Ag85B and Hsp70 were evaluated in C57Bl/6 mice (FIG. 4A). Mice were monitored for clinical signs such as inflammation at the site of vaccine injection, depression, or inappetence. No clinical signs were observed in all groups over the course of 15 weeks. Although QTAP-mRNA vaccinated mice showed hair loss at the posterior and dorsal regions, we observed no clinical severities in these groups. The weight of mice vaccinated with either QTAP only or QTAP-mRNA groups did not differ from PBS inoculated mice (FIG. 4B). ELISA titers in blood indicated that QTAP-mRNA (Ag85B+Hsp70) elicited significant levels of IFN-γ, TNF-α, and IL-17 specific cytokines compared to PBS control group. Interestingly, the QTAP-only group showed elevated cytokine levels relative to the PBS group suggesting that QTAP by itself is immunogenic (FIGS. 4C-E). Overall, these findings demonstrate the adjuvant effect of QTAP alone which is amplified by the encapsulated mRNA. Histopathological analysis of the lungs shows no pathological damage (data not shown). At 4 weeks post-final vaccination, we quantified the percentage of CD4+ and CD8+ T cells in the lungs and spleen of mice from all groups and analyzed the population of Th-1 (IFN-γ, TNF-α, IL-2) and Th-17 (IL-17A) cytokine-producing T cells by flow cytometry (FIG. 5). The results indicate that QTAP and QTAP-mRNA elicit significantly higher proportions of CD 4 T cells secreting pro-inflammatory cytokines in mice compared to the PBS group. However, there were no significant levels of these cytokines in CD8 T cells (data not shown). Interestingly, humoral immune response characterized by the presence of IgG antibodies were detected at significantly higher levels in the sera of QTAP-mRNA immunized mice compared to the control groups. This increase in IgG response was detected as early as 5 weeks after the second vaccine dose and up to at least 4 weeks post-challenge (FIGS. 5E, F). Overall, these results suggest that QTAP encapsulating modified mRNA encoding mycobacterial Ag85B and Hsp70 is safe and immunogenic in mice.

3.5 Immunization with QTAP Nanovaccine Protects Mice Against Mycobacterium avium ss. Hominissuis Infection

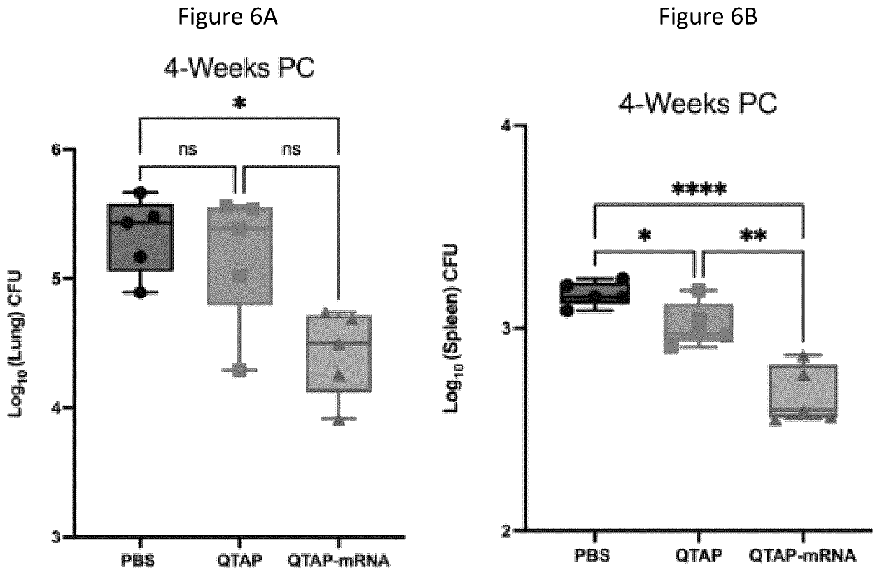

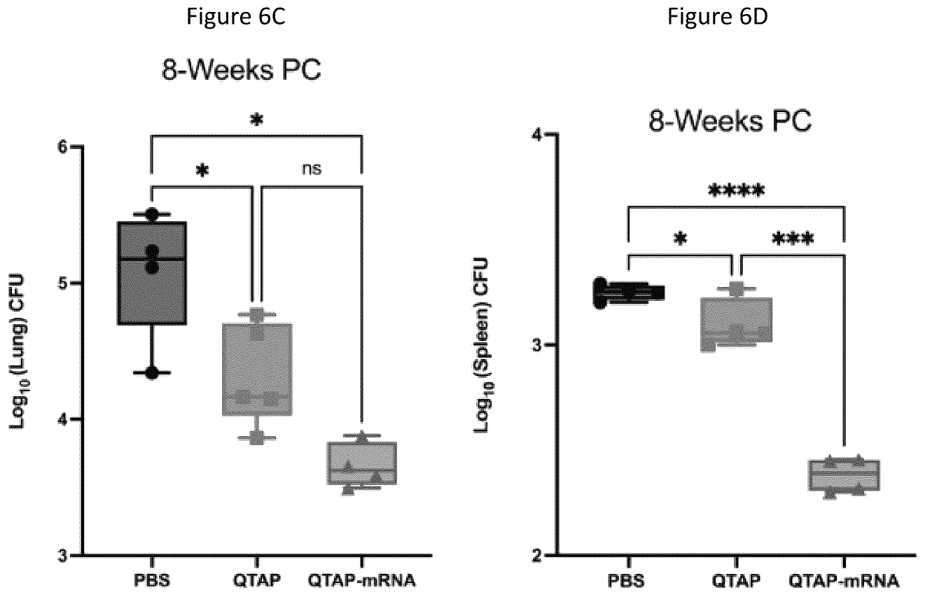

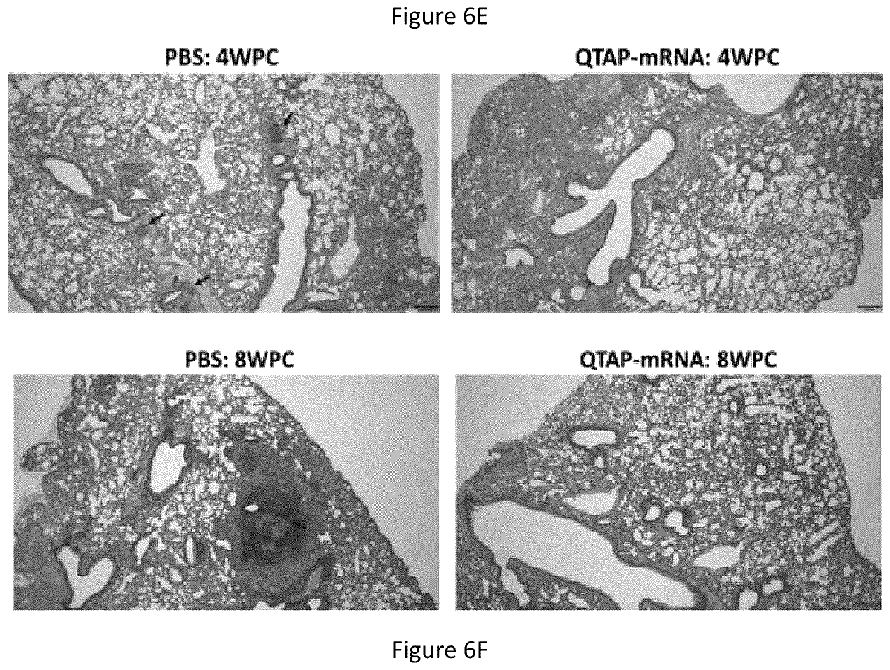

To investigate the protective efficacy of the novel QTAP nanovaccine, we analyzed the bacterial burden and histopathological changes in vaccinated and challenged mice at different times post-challenge (FIG. 4A). Bacterial counts showed that vaccination of mice with QTAP-mRNA resulted in a significant reduction in bacterial burden in the lungs and spleen at both timepoints (FIGS. 6A-D) with little pathological damages of the lung airways (FIGS. 6E, F). In unvaccinated mice, we observed pre-granuloma structures at 4 weeks post-infection and fully formed granuloma-like structures at 8 weeks post-challenge with extensive damage to the lung airways. Analysis of the spleen showed no noticeable pathological damage in both groups.

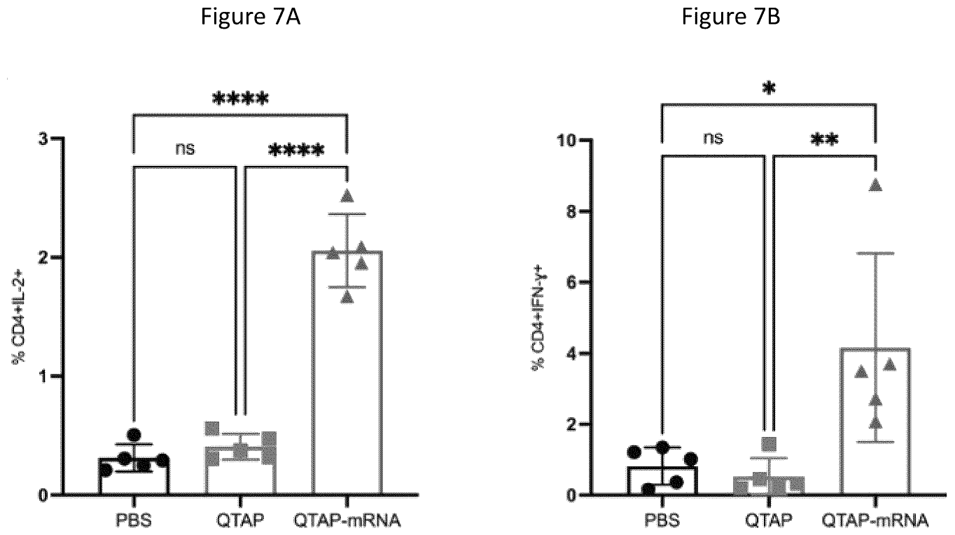

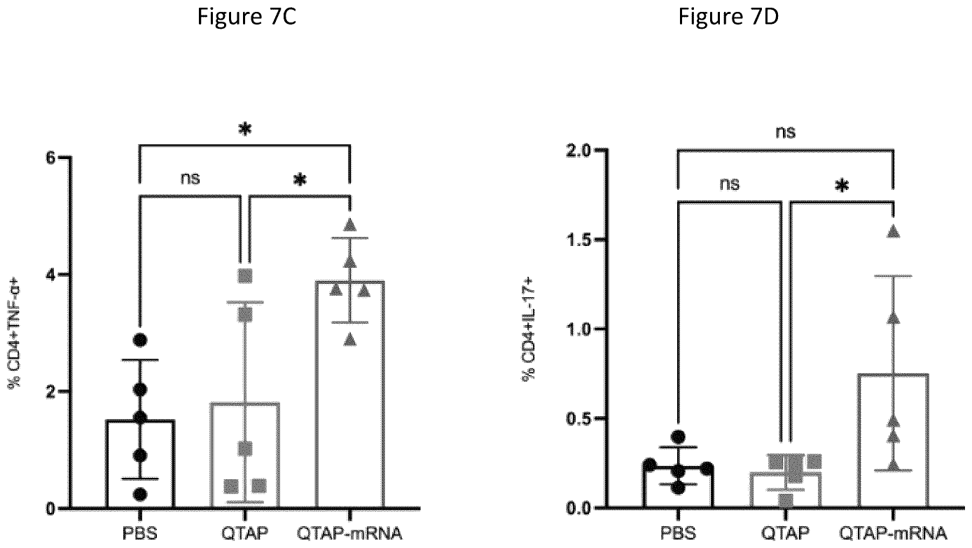

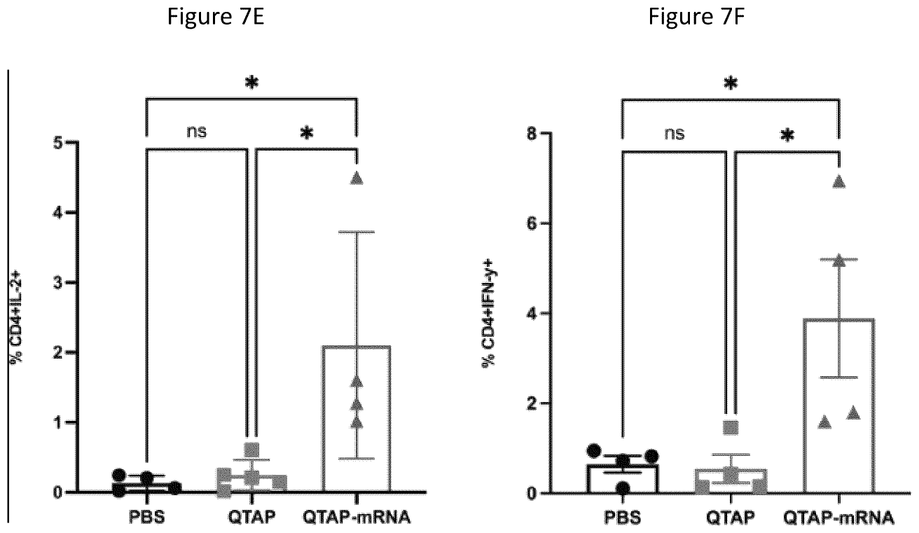

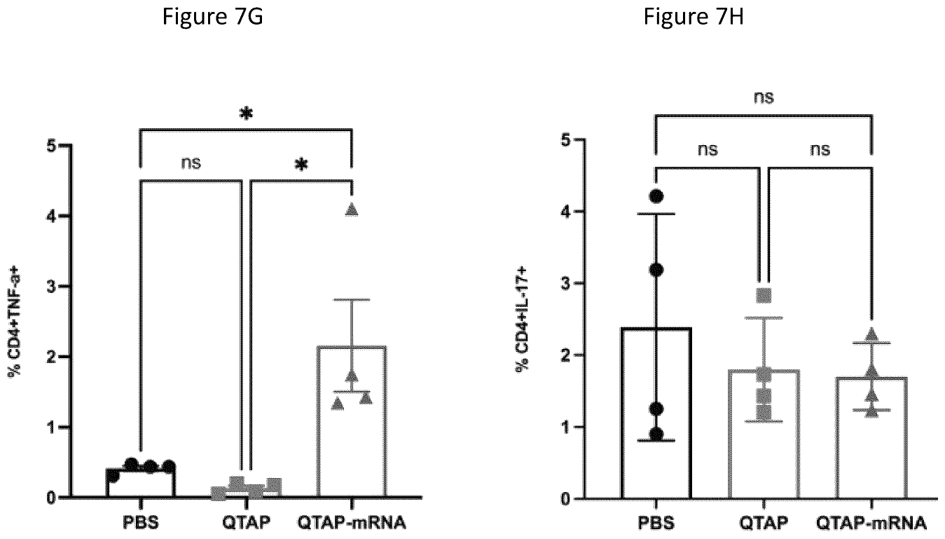

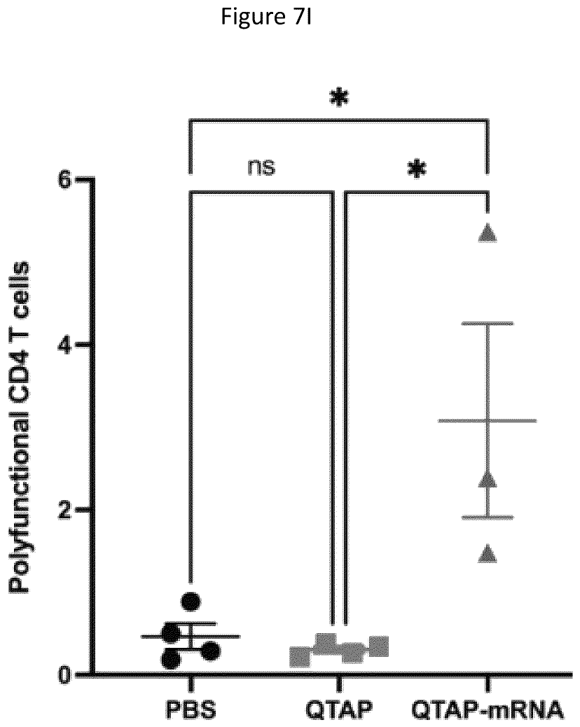

To characterize the immune response of protection, vaccinated mice challenged with M. ah resulted in a higher number of CD4+ T-cells secreting IFN-γ, IL-2, TNF-α, and IL-17A cytokines compared to PBS and QTAP-only groups at 4 weeks post-challenge (FIGS. 7A-D). At 8 weeks post-challenge, CD4+ and CD8+ (data not shown) T-cells secreting IFN-γ, TNF-α, and IL-2, remain significantly higher in the vaccinated group with no apparent difference in IL-17A among the groups (FIGS. 7E-H). A similar profile for CD8+ T cell response was detected at this timepoint (data not shown). Interestingly, QTAP-mRNA immunized mice had poly-functional CD4 T cells secreting IFN-γ, IL-2, and TNF-α in only their lungs at 8 weeks post-challenge (FIG. 71). These findings suggest that the novel QTAP-mRNA nanovaccine is protective against M. ah infection in mice.

### 4 Discussion

Recently, mRNA vaccines have attracted great attention since their successful use to combat the COVID-19 pandemic (44, 45). Despite this success, it remains unknown whether the same approach can be used to target more challenging pathogens such as mycobacteria. Unlike SARS-CoV-2, mycobacterial pathogens use complex pathogenic mechanisms consisting of a plethora of virulence factors for evasion of host immune pathways that enable them to either cause disease or remain persistent in the lungs (46, 47). This presents a challenge for designing effective vaccines against mycobacteria. However, the mRNA vaccine technology allows for the careful design of vaccine constructs with the flexibility to include different antigens to target multiple pathogenic pathways (44, 48). We hypothesize that vaccines targeting mycobacterial pathogens should be rationally designed to include multiple antigens to match the complexity of the intracellular life cycle of the bacteria. Indeed, it has been previously shown that RNA vaccine (repRNA-ID91/ID91+GLA-SE) encoding four M. ah antigens produced significant cellular and humoral immune responses leading to reduced bacterial burden (33). The same group showed that repRNA-ID91/ID91+GLA-SE provided similar protection against M. tb infection in mice (29). However, the highest protection generated by this vaccine was when it is used in a prime (RNA)-boost (protein) regimen (29, 33). Beyond design, mRNA vaccines require sufficient immunostimulatory adjuvants to achieve optimal protective efficacy (49, 50). A recent study has shown that those LNPs made from DOTAP, 1,2-dipalmitoyl-sn-glycero-3-phosphocholine (DPPC), and cholesterol alone failed to provoke inflammatory responses such as pro-inflammatory cytokine production and inflammatory cell infiltration in mice (51). On the other hand, LNPs made with recombinant hemagglutinin (HA) and neuraminidase proteins induce inflammatory responses for influenza seasonal Flu vaccines (51). These findings demonstrate the limitation of LNPs alone in eliciting optimal immune response while also highlighting the importance of additional adjuvants in eliciting pro-inflammatory immune responses which is a pre-requisite for protection against mycobacterial infections (52). The repRNA-ID91/ID91+GLA-SE vaccine-mediated cellular and humoral immune response leading to protective immunity against both M. ah and M. tb is associated with the GLA-SE adjuvant (29, 33). The primary goal of the studies reported here was to evaluate the novel QTAP nanoadjuvant platform for the development of effective mRNA vaccines targeting M. ah infection in mice. The initial studies focused on the mRNA delivery capability of QTAP nanoadjuvant to efficiently entrap and deliver mRNA in cells and elicit an inflammatory profile suitable for the control of mycobacteria in mice.

We show that QTAP nanoadjuvant complexed with mRNA forms nanoparticles (NPs). The physical parameters of QTAP NPs encapsulating mRNA (˜75 nm, positively charged) are consistent with previous findings suggesting that positively charged NPs with a size range of 50-150 nm are suitable for induction of sufficient immune response in both mice and non-human primates (53, 54). Under in vitro conditions designed to mimic cellular environmental conditions, QTAP NPs released mRNA in a sustained manner over a long period of time. Indeed, previous studies have demonstrated that prolonged release is important for sustained induction of immune response over a long period and enables sufficient lymphocyte activation and proliferation with cytokine induction (55). Additionally, when stored at different temperatures, QTAP NPs protect mRNA from degradation demonstrated by the expression of encoded proteins in cells after transfection (56). Most FDA-approved mRNA vaccines and those in clinical trials require extremely low temperatures for storage (57, 58). This limits their application in areas of the world that harbors the greatest burden of infectious diseases with high temperature and limited access to cold storage. We showed that QTAP NPs can protect mRNA at even higher temperatures (4-20° C.) than the reported temperature requirements of current FDA-approved LNP/mRNA vaccines (57, 58).

QTAP nanoadjuvant encapsulating modified mRNA encoding either Luc, GFP, Ag85B, or Hsp70 gene showed higher protein expression and transfection efficiency compared to DOTAP-mRNA. In BHK cells modified mRNA delivered by QTAP NPs showed higher protein expression similar to previous reports (42, 59). Additionally, we showed that the presence of Quil-A in the LNP/mRNA showed a more than 200% increase in GFP+ cells in QTAP-transfected BHK cells. As previously reported, mRNA-based vaccine immunogenicity and protective efficacy are dependent on the amount of mRNA-derived protein antigens (55). Moreover, we showed that macrophages exposed to QTAP nanoadjuvant encapsulating mRNA-Ag85B exhibit elevated induction of NLRP3 inflammasomes, NF-kb, and My-D88. We observed that QTAP alone did not lead to any increase in CD80 expression in macrophages. However, although statistically insignificant, we noticed that QTAP-treated macrophages have a slight increase in expression of CD86 compared to the negative control. We expected QTAP-mRNA to have higher expression of these co-stimulatory molecules because mRNA by itself is immunogenic. Secondly, QTAP alone showed a slight increase in activation of both NF-kb and My-D88 compared to both negative and DOTAP-only controls. Although statistically insignificant, it is not clear whether such a slight increase in NF-kb and My-D88 activation has any biological significance. LNP-based activation of effector immune cells has been shown to be required for the induction of effective immune response against pathogens (60-62). Due to these reasons, the DOTAP-mRNA group is not included in the animal immunization and challenge experiments since it has minimal transfection efficiency and macrophage activation ability.

Mouse vaccination studies indicated that QTAP-mRNA encoding Ag85B and Hsp70 is safe and highly immunogenic. A dose of 15 μg in a 3-dose immunization regimen, resulted in no significant changes in mouse weight. This immunization regimen elicited robust cell and humoral-mediated immune responses. We observed that immunization of mice with QTAP-mRNA encoding Ag85B and Hsp70 produced both Th-1 and Th-17 immune responses demonstrated by elevated secretion of proinflammatory cytokines IL-2, IFN-γ, TNF-α, and IL-17 by these T cells. Previous studies have shown that the humoral immune response plays a crucial role in the protection of the host against mycobacterial infections. The QTAP-mRNA vaccine elicited a robust antibody response characterized by elevated IgG levels detected in mouse sera very early after immunization and lasting even after infection. Additionally, we showed that the vaccine was not associated with any pathology in the lungs compared to the PBS control group. However, a mild increase in lung infiltrating lymphocytes was seen with no apparent pathological damage of the airway in the vaccinated group. Previously, protein vaccine boosted with RNA against M. ah challenge in mice showed reduced lesions in the lung compared to naïve control (33). However, the vaccinated mice had more than 40% of their lungs affected by lesions (33). In the case of QTAP-mRNA, we did not observe such widespread lesions in the lungs even though the vaccine contains only two mycobacterial antigens.

Interestingly, immunized mice challenged with M. ah demonstrated significantly reduced bacterial burden in both the lungs and spleen at both 4- and 8-weeks post-challenge. The T cell response profile at these time points was similar to the pre-challenge profile. At 4 weeks post-infection, we observed a significant increase in lung infiltrating IL-2, IFN-γ, TNF-α, and IL-17 secreting CD4 but not CD8 T cells in the vaccinated group. These findings are like the results of previous RNA vaccines against both M. ah and M. tb in mice (29, 33). In M. tb and M. ah infections, continuous exposure to the bacteria causes exhaustion in CD8 T cells thereby inhibiting their cytokine secretory capability (63-65). From these findings, in the presence of active CD4 T cell response to M. ah in vaccinated mice, the absence of CD8 T cell response did not seem to stop the reduction in infection burden. However, we cannot disqualify the absence of CD8 T cell response as a limiting factor of M. ah control. We hypothesize that augmentation of CD8 T cell response will strengthen the protective efficacy of mRNA vaccines against mycobacterial pathogens. Further studies using CD8 T cell depletion will help to decipher the role of CD8 T cells in the QTAP-mRNA nanovaccines mediated protection against M. ah. At 8 weeks post-challenge, a similar T cell response was observed in CD4 T cells. However, we noticed that IL-17 was highly elevated in the PBS group unlike at 4 weeks post-challenge, while IL-2, IFN-γ, TNF-α, and IL-17 cytokines maintained similar levels in the vaccinated mice from 4 to 8 weeks. This profile might be due to the Th-1/Th-17 imbalance previously reported in active mycobacterial infections where a shift towards excessive IL-17 response causes extensive neutrophil recruitment and tissue damage (66-69). Indeed, we observed extensive pathological damages only in the lungs of unvaccinated mice with large granuloma structures. Also, unlike the 4-week post-challenge time-point, CD8 T cell responses in the vaccinated mice were enhanced at 8 weeks post-challenge characterized by a significant increase in IL-2, IFN-γ, TNF-α cytokines levels. However, the level of cytokines secreted by CD4 T cells was higher than CD8 T cells. This observation supports previous findings that in the case of mycobacterial infections such as M. tb, the primary immune response responsible for protective immunity against infection is CD4 T cell-mediated (70) with evidence of poor CD8 T cell-mediated protection (71). Recent studies have shown that CD8 T cells recognize M. tb and have cytolytic functions and produce inflammatory cytokines highlighting their important role in M. tb control (72, 73). In other studies, CD4 T cells have been shown to help prevent CD8 T cell exhaustion during M. tb infection, and that in the absence of CD4 T cells, CD8 T cell-mediated protection is underestimated (63, 74). These findings demonstrate the importance of both classes of T cells and demonstrate the synergy between CD4 and CD8 T cells in the control of M. tb infection. In our case, we showed that the novel QTAP-mRNA vaccine elicits predominantly CD4 T cell response during the early phases of infection whereas the late phase of infection is characterized by both CD4 and CD8 T cell response in vaccinated mice. Additionally, at 8 weeks post-infection, we demonstrated more than 1.5 log reduction of bacterial CFUs in the lungs of the vaccinated mice.

Overall, we show that the use of the novel QTAP nanoadjuvant for the delivery of mRNA vaccine constructs targeting M. ah is highly effective in eliciting protective immunity. The QTAP nanoadjuvant is a promising system for the effective delivery of mRNA vaccine constructs for both in vitro and in vivo models with the added value of thermostability at higher temperatures. While LNP-mRNA vaccines targeting mycobacterial pathogens may prove effective, QTAP nanoadjuvanted mRNA-based vaccines will likely provide long-lasting immunity.

### 5. REFERENCES

1. Stenler, S., P. Blomberg, and C. I. Smith, Safety and efficacy of DNA vaccines: plasmids vs. minicircles. Hum Vaccin Immunother, 2014. 10 (5): p. 1306-8.
2. Wang, M., et al., Protective efficacy induced by Eimeria maxima rhomboid-like protein 1 against homologous infection. Front Vet Sci, 2022. 9: p. 1049551.
3. Rosenblum, H. G., et al., Safety of mRNA vaccines administered during the initial 6 months of the US COVID-19 vaccination programme: an observational study of reports to the Vaccine Adverse Event Reporting System and v-safe. Lancet Infect Dis, 2022. 22 (6): p. 802-812.
4. Niesen, M. J. M., et al., Surveillance of Safety of 3 Doses of COVID-19 mRNA Vaccination Using Electronic Health Records. JAMA Netw Open, 2022. 5 (4): p. e227038.
5. Pulendran, B., S. A. P, and D. T. O'Hagan, Emerging concepts in the science of vaccine adjuvants. Nat Rev Drug Discov, 2021. 20 (6): p. 454-475.
6. O'Hagan, D. T., R. N. Lodaya, and G. Lofano, The continued advance of vaccine adjuvants-‘we can work it out’. Semin Immunol, 2020. 50: p. 101426.
7. Cajaraville, A., et al., Evaluation of Two Adjuvant Formulations for an Inactivated Yellow Fever 17DD Vaccine Candidate in Mice. Vaccines (Basel), 2022. 11 (1).
8. Coban, C., et al., Novel strategies to improve DNA vaccine immunogenicity. Curr Gene Ther, 2011. 11 (6): p. 479-84.
9. Smith, L. R., et al., Phase 1 clinical trials of the safety and immunogenicity of adjuvanted plasmid DNA vaccines encoding influenza A virus H5 hemagglutinin. Vaccine, 2010. 28 (13): p. 2565-72.
10. Doener, F., et al., RNA-based adjuvant CV8102 enhances the immunogenicity of a licensed rabies vaccine in a first-in-human trial. Vaccine, 2019. 37 (13): p. 1819-1826.
11. Heidenreich, R., et al., A novel RNA-based adjuvant combines strong immunostimulatory capacities with a favorable safety profile. Int J Cancer, 2015. 137 (2): p. 372-84.
12. Wilson, B. and K. M. Geetha, Lipid nanoparticles in the development of mRNA vaccines for COVID-19. J Drug Deliv Sci Technol, 2022. 74: p. 103553.
13. Ho, W., et al., Next-Generation Vaccines: Nanoparticle-Mediated DNA and mRNA Delivery. Adv Healthc Mater, 2021. 10 (8): p. e2001812.
14. Ndeupen, S., et al., The mRNA-LNP platform's lipid nanoparticle component used in preclinical vaccine studies is highly inflammatory. iScience, 2021. 24 (12): p. 103479.
15. Hald Albertsen, C., et al., The role of lipid components in lipid nanoparticles for vaccines and gene therapy. Adv Drug Deliv Rev, 2022. 188: p. 114416.
16. Eygeris, Y., et al., Chemistry of Lipid Nanoparticles for RNA Delivery. Acc Chem Res, 2022. 55 (1): p. 2-12.
17. Kon, E., U. Elia, and D. Peer, Principles for designing an optimal mRNA lipid nanoparticle vaccine. Curr Opin Biotechnol, 2022. 73: p. 329-336.
18. Kranz, L. M., et al., Systemic RNA delivery to dendritic cells exploits antiviral defence for cancer immunotherapy. Nature, 2016. 534 (7607): p. 396-401.
19. Krienke, C., et al., A noninflammatory mRNA vaccine for treatment of experimental autoimmune encephalomyelitis. Science, 2021. 371 (6525): p. 145-153.
20. T. Pilishvili, R. G., K. E. Fleming-Dutra, J. L. Farrar, N. M. Mohr, D. A. Talan, et al., Effectiveness of mRNA Covid-19 Vaccine among U.S. Health Care Personnel. The New England Journal of Medicine, 2021. 122 (3): p. 13.
21. Lauring, A. S., et al., Clinical severity of, and effectiveness of mRNA vaccines against, covid-19 from omicron, delta, and alpha SARS-CoV-2 variants in the United States: prospective observational study. BMJ, 2022. 376: p. e069761.
22. Laurens, M. B., RTS,S/AS01 vaccine (Mosquirix): an overview. Hum Vaccin Immunother, 2020. 16 (3): p. 480-489.
23 Maltz, F. and B. Fidler, Shingrix: A New Herpes Zoster Vaccine. P T, 2019. 44 (7): p. 406-433.
24 Lacaille-Dubois, M. A., Updated insights into the mechanism of action and clinical profile of the immunoadjuvant QS-21: A review. Phytomedicine, 2019. 60: p. 152905.
25. Fan, X. Y. and D. B. Lowrie, Where are the RNA vaccines for TB? Emerg Microbes Infect, 2021. 10 (1): p. 1217-1218.
26 Sharma, S. K. and V. Upadhyay, Epidemiology, diagnosis & treatment of non-tuberculous mycobacterial diseases. Indian J Med Res, 2020. 152 (3): p. 185-226.
27. Adjemian, J., et al., Epidemiology of Nontuberculous Mycobacteriosis. Semin Respir Crit Care Med, 2018. 39 (3): p. 325-335.
28 Delogu, G., et al., DNA vaccination against tuberculosis: expression of a ubiquitin-conjugated tuberculosis protein enhances antimycobacterial immunity. Infect Immun, 2000. 68 (6): p. 3097-102.
29 Larsen, S. E., et al., An RNA-Based Vaccine Platform for Use against Mycobacterium tuberculosis. Vaccines (Basel), 2023. 11 (1).
30 Maurya, S. K., et al., A multiple T cell epitope comprising DNA vaccine boosts the protective efficacy of Bacillus Calmette-Guerin (BCG) against Mycobacterium tuberculosis. BMC Infect Dis, 2020. 20 (1): p. 677.
31. Ghandadi, M., An Immunoinformatic Strategy to Develop New Mycobacterium tuberculosis Multi-epitope Vaccine. Int J Pept Res Ther, 2022. 28 (3): p. 99.
32. Chatterjee, N., et al., Scrutinizing Mycobacterium tuberculosis membrane and secretory proteins to formulate multiepitope subunit vaccine against pulmonary tuberculosis by utilizing immunoinformatic approaches. Int J Biol Macromol, 2018. 118 (Pt A): p. 180-188.
33 Rais, M., et al., Immunogenicity and protection against Mycobacterium avium with a heterologous RNA prime and protein boost vaccine regimen. Tuberculosis (Edinb), 2023. 138: p. 102302.
34. Ali, Z. I., et al., Genotypic analysis of nontuberculous mycobacteria isolated from raw milk and human cases in Wisconsin. J Dairy Sci, 2021. 104 (1): p. 211-220.
35. Marcus, S. A., H. Steinberg, and A. M. Talaat, Protection by novel vaccine candidates, Mycobacterium tuberculosis DeltamosR and DeltaechA7, against challenge with a Mycobacterium tuberculosis Beijing strain. Vaccine, 2015. 33 (42): p. 5633-5639.
36 Kingstad-Bakke, B. A., et al., Effective mosaic-based nanovaccines against avian influenza in poultry. Vaccine, 2019. 37 (35): p. 5051-5058.
37 Hildebrand, R. E., et al., Superinfection with SARS-CoV-2 Has Deleterious Effects on Mycobacterium bovis BCG Immunity and Promotes Dissemination of Mycobacterium tuberculosis. Microbiol Spectr, 2022. 10 (5): p. e0307522.
38. Ashizawa, K., [Nanosize Particle Analysis by Dynamic Light Scattering (DLS)]. Yakugaku Zasshi, 2019. 139 (2): p. 237-248.
39. Green, M. R. and J. Sambrook, Quantifying and Storing RNA. Cold Spring Harb Protoc, 2020. 2020 (3): p. 101709.
40. Desjardins, P. and D. Conklin, NanoDrop microvolume quantitation of nucleic acids. J Vis Exp, 2010 (45).
41. Chandrasekar, S. S., et al., A DNA Prime and MVA Boost Strategy Provides a Robust Immunity against Infectious Bronchitis Virus in Chickens. Vaccines (Basel), 2023. 11 (2).
42 Kariko, K., et al., Incorporation of pseudouridine into mRNA yields superior nonimmunogenic vector with increased translational capacity and biological stability. Mol Ther, 2008. 16 (11): p. 1833-40.
43. Kauffman, K. J., et al., Efficacy and immunogenicity of unmodified and pseudouridine-modified mRNA delivered systemically with lipid nanoparticles in vivo. Biomaterials, 2016. 109: p. 78-87.
44 Hajissa, K. and A. Mussa, Positive aspects of the mRNA platform for SARS-CoV-2 vaccines. Hum Vaccin Immunother, 2021. 17 (8): p. 2445-2447.
45. Jain, S., et al., Messenger RNA-based vaccines: Past, present, and future directions in the context of the COVID-19 pandemic. Adv Drug Deliv Rev, 2021. 179: p. 114000.
46. Delogu, G., et al., Mycobacterium tuberculosis virulence: insights and impact on vaccine development. Future Microbiol, 2015. 10 (7): p. 1177-94.
47 Mascart, F. and C. Locht, Integrating knowledge of Mycobacterium tuberculosis pathogenesis for the design of better vaccines. Expert Rev Vaccines, 2015. 14 (12): p. 1573-85.
48. Ilyichev, A. A., et al., mRNA technology as one of the promising platforms for the SARS-CoV-2 vaccine development. Vavilovskii Zhurnal Genet Selektsii, 2020. 24 (7): p. 802-807.
49 Pardi, N., M. J. Hogan, and D. Weissman, Recent advances in mRNA vaccine technology. Curr Opin Immunol, 2020. 65: p. 14-20.
50. McDonald, I., et al., Comparative systematic review and meta-analysis of reactogenicity, immunogenicity and efficacy of vaccines against SARS-CoV-2. NPJ Vaccines, 2021. 6 (1): p. 74.
51. Shirai, S., et al., Lipid Nanoparticle Acts as a Potential Adjuvant for Influenza Split Vaccine without Inducing Inflammatory Responses. Vaccines (Basel), 2020. 8 (3).
52. Liu, C. H., H. Liu, and B. Ge, Innate immunity in tuberculosis: host defense vs pathogen evasion. Cell Mol Immunol, 2017. 14 (12): p. 963-975.
53. Haseda, Y., et al., Microfluidic-prepared DOTAP nanoparticles induce strong T-cell responses in mice. PLOS One, 2020. 15 (1): p. e0227891.
54. Hassett, K. J., et al., Impact of lipid nanoparticle size on mRNA vaccine immunogenicity. J Control Release, 2021. 335: p. 237-246.
55 Pardi, N., et al., mRNA vaccines—a new era in vaccinology. Nat Rev Drug Discov, 2018. 17 (4): p. 261-279.
56. Kumar, R., et al., Thermostable vaccines: an innovative concept in vaccine development. Expert Rev Vaccines, 2022. 21 (6): p. 811-824.
57. Uddin, M. N. and M. A. Roni, Challenges of Storage and Stability of mRNA-Based COVID-19 Vaccines. Vaccines (Basel), 2021. 9 (9).
58. Kloczewiak, M., et al., A Biopharmaceutical Perspective on Higher-Order Structure and Thermal Stability of mRNA Vaccines. Mol Pharm, 2022. 19 (7): p. 2022-2031.
59. Anderson, B. R., et al., Incorporation of pseudouridine into mRNA enhances translation by diminishing PKR activation. Nucleic Acids Res, 2010. 38 (17): p. 5884-92.
60. Shi, J., et al., Delivery of mRNA for regulating functions of immune cells. J Control Release, 2022. 345: p. 494-511.
61. Lonez, C., et al., Cationic lipid nanocarriers activate Toll-like receptor 2 and NLRP3 inflammasome pathways. Nanomedicine, 2014. 10 (4): p. 775-82.
62. Nelson, J., et al., Impact of mRNA chemistry and manufacturing process on innate immune activation. Sci Adv, 2020. 6 (26): p. eaaz6893.
63. Lu, Y. J., et al., CD4 T cell help prevents CD8 T cell exhaustion and promotes control of Mycobacterium tuberculosis infection. Cell Rep, 2021. 36 (11): p. 109696.
64. Lombardi, A., et al., T-Cell Exhaustion in Mycobacterium tuberculosis and Nontuberculous Mycobacteria Infection: Pathophysiology and Therapeutic Perspectives. Microorganisms, 2021. 9 (12).
65. Jayaraman, P., et al., TIM3 Mediates T Cell Exhaustion during Mycobacterium tuberculosis Infection. PLOS Pathog, 2016. 12 (3): p. e1005490.
66. Torrado, E. and A. M. Cooper, IL-17 and Th17 cells in tuberculosis. Cytokine Growth Factor Rev, 2010. 21 (6): p. 455-62.
67. Luo, J., et al., Imbalance of Th17 and Treg in peripheral blood mononuclear cells of active tuberculosis patients. Braz J Infect Dis, 2017. 21 (2): p. 155-161.
68. Marin, N. D., et al., Reduced frequency of memory T cells and increased Th17 responses in patients with active tuberculosis. Clin Vaccine Immunol, 2012. 19 (10): p. 1667-76.
69. Damsker, J. M., A. M. Hansen, and R. R. Caspi, Th1 and Th17 cells: adversaries and collaborators. Ann N Y Acad Sci, 2010. 1183: p. 211-21.
70. Lo, C. Y., et al., Increased Th1 Cells with Disease Resolution of Active Pulmonary Tuberculosis in Non-Atopic Patients. Biomedicines, 2021. 9 (7).
71. Yang, J. D., et al., Mycobacterium tuberculosis-specific CD4+ and CD8+ T cells differ in their capacity to recognize infected macrophages. PLOS Pathog, 2018. 14 (5): p. e1007060.
72. McMurtrey, C., et al., T cell recognition of Mycobacterium tuberculosis peptides presented by HLA-E derived from infected human cells. PLOS One, 2017. 12 (11): p. e0188288.
73. Harriff, M. J., et al., HLA-E Presents Glycopeptides from the Mycobacterium tuberculosis Protein MPT32 to Human CD8 (+) T cells. Sci Rep, 2017. 7 (1): p. 4622.
74. Wang, X., et al., Protection against Mycobacterium tuberculosis infection offered by a new multistage subunit vaccine correlates with increased number of IFN-gamma+IL-2+CD4+ and IFN-gamma+CD8+ T cells. PLOS One, 2015. 10 (3): p. e0122560.

### Example 2-Immune Responses Generated by a Novel Nanoadjuvant System Against Mycobacterium avium ss. Hominissuis

Reference is made to the abstract: Touray et al., “Immune responses generated by a novel nanoadjuvant system against Mycobacterium avium ss. hominissuis” the content of which is incorporated herein by reference in its entirety.

Avian influenza virus (AIV) continues to pose a significant threat to poultry health and global food security. In this study, we aimed to enhance the immunogenicity of a mosaic DNA vaccine against AIV by incorporating a novel nanoadjuvant dubbed QTAP comprising Quil-A and 1,2-dioleoyl-3-trimethylammonium-propane (DOTAP), while ensuring its safety through monitoring of chicken weight post-vaccination. The mosaic vaccine design encompassed conserved regions of the hemagglutinin (HA) protein from diverse AIV strains to broaden immune recognition, and the nanoadjuvant was utilized to augment the immune response and improve vaccine stability. Chickens were immunized twice via the subcutaneous route, and serum samples were collected post-vaccination to assess hemagglutination inhibition (HI) titers. Additionally, chicken weights were monitored throughout the study as an indicator of vaccine safety. Preliminary findings revealed a substantial increase in HI titers without any significant changes in chicken weight, demonstrating the safety and immunogenicity of the nanoadjuvanted mosaic DNA vaccine. These results underscore the potential of this innovative vaccine formulation as a promising and safe prophylactic strategy against AIV infection in chickens. Further research is ongoing to evaluate the vaccine's protective efficacy, longevity of immunity, and suitability for commercial application in poultry production.

### INTRODUCTION

Avian influenza virus (AIV) represents a dual threat to both the global poultry industry and public health, imposing substantial economic burdens and highlighting its zoonotic potential [1, 2]. The continuous emergence of new AIV strains with varying pathogenicity and transmissibility poses significant challenges to animal health, food security, and economic stability, leading to devastating losses in poultry production and trade restrictions imposed by affected countries [3]. Economically, AIV outbreaks can result in massive losses due to poultry mortality, reduced egg production, trade embargoes, and the costs associated with disease control measures and eradication campaigns [4]. In addition to immediate economic impacts, the long-term consequences of AIV outbreaks can include reduced consumer confidence in poultry products, market instability, and the potential for negative repercussions on the broader agricultural sector and national economies [5]. These economic ramifications underscore the critical need for effective and timely interventions to prevent and control AIV outbreaks in poultry populations.

Beyond its economic implications, AIV also possesses zoonotic potential, representing a significant public health concern [1, 6]. While most AIV strains predominantly circulate among birds, certain subtypes, such as H5N1 and H7N9, have demonstrated the ability to infect humans, leading to severe respiratory illness, hospitalizations, and fatalities [7]. The zoonotic transmission of AIV underscores the interconnectedness of animal and human health and highlights the importance of implementing comprehensive One Health approaches to disease surveillance, prevention, and control.

Traditional vaccination strategies, including inactivated and live-attenuated vaccines, have been the cornerstone of AIV control in poultry; however, these approaches often exhibit limitations in terms of cross-protection, safety, and scalability [8]. In recent years, the development of DNA vaccines has garnered increasing attention due to their potential advantages, including rapid production, stability, and the ability to induce robust cellular and humoral immune responses [9, 10]. Mosaic DNA vaccines, designed to incorporate conserved regions from multiple viral strains, offer a promising approach to broaden immune recognition and enhance vaccine efficacy against diverse AIV variants [11, 12].

Despite these advancements, the immunogenicity of DNA vaccines can often be suboptimal, necessitating the incorporation of adjuvants or delivery systems to improve antigen presentation and immune stimulation [13, 14]. Nanotechnology-based adjuvants have emerged as a promising tool to augment the immunogenicity of vaccines by enhancing antigen uptake, promoting sustained antigen release, and modulating immune responses [14-16]. The integration of nanoadjuvants with mosaic DNA vaccines could potentially amplify their protective efficacy against AIV while maintaining safety and reducing the frequency of administration.

In this context, the present study aims to develop and evaluate a nanoadjuvanted mosaic DNA vaccine against AIV infection in chickens. We hypothesize that the integration of a novel nanoadjuvant with a mosaic DNA vaccine will enhance the immune response, broaden the spectrum of protection, and maintain the safety profile of the vaccine as evidenced by no changes in chicken weight post-vaccination. Through comprehensive immunogenicity and safety assessments, this research seeks to contribute to the advancement of effective, safe, and scalable vaccination strategies for the control and prevention of AIV in poultry populations, thereby addressing both the economic and zoonotic challenges associated with this pervasive viral pathogen.

### Methods

### Vaccine Design and Preparation

A mosaic DNA vaccine targeting conserved regions of Avian influenza virus (AIV) hemagglutinin (HA) was designed using bioinformatics tools to maximize immune recognition and cross-protection against diverse AIV strains. The synthetic DNA sequences were codon optimized for expression in chickens and cloned into a suitable plasmid vector under the control of a CMV early enhancer/chicken β actin (CAG) promoter. E. coli DH5-alpha were transformed with the plasmid DNA and cultured on an ampicillin LB plate for selection. Single colonies were picked and grown in 5 ml LB broth at 37° C. while shaking. After 24 hrs, the bacterial culture was transferred to 1 L LB broth and cultured at 37° C. while shaking for 48 hrs. Plasmid DNA was isolated using PureLink™ HiPure Expi Plasmid Gigaprep Kit (ThermoFisher Scientific, Waltham, MA, USA) and stored in endotoxin free tubes at −20° C. until use.

### Nanoadjuvant Synthesis and Characterization

The nanoadjuvant was synthesized using a previously described nanoparticle formulation [17], comprising biodegradable and biocompatible materials selected for their safety profile and adjuvant properties. The physicochemical properties of the nanoadjuvant, including size, zeta potential, and morphology, were characterized using dynamic light scattering (DLS), zeta potential analysis, and transmission electron microscopy (TEM), respectively.

### Animal Immunization and Sample Collection

Specific pathogen-free (SPF) chickens were obtained from a certified supplier and housed under standard laboratory conditions with ad libitum access to water and feed. Chickens were randomly divided into experimental groups and immunized twice with a two-week interval via the subcutaneous route with the nanoadjuvanted mosaic DNA (100 μg) vaccine or control formulations.

Serum samples were collected from each chicken prior to each immunization and two weeks after the final vaccination to assess hemagglutination inhibition (HI) titers and monitor changes in chicken weight as an indicator of vaccine safety. Blood samples were centrifuged at 3,000×g for 10 minutes to obtain serum, which was stored at −20° C. until further analysis.

### Immunogenicity Assessment

HI titers were determined using standard protocols with homologous and heterologous AIV strains as antigens. Briefly, serially diluted chicken serum samples were mixed with a standardized amount of AIV antigen, followed by the addition of Chicken red blood cells (RBCs) and incubation at room temperature for 30 minutes. HI titers were expressed as the reciprocal of the highest serum dilution that completely inhibited hemagglutination.

### Safety Evaluation

Changes in chicken weight were monitored throughout the study as an indicator of vaccine safety. Individual chicken weights were recorded weekly and compared between experimental groups to assess any potential adverse effects or alterations in growth rates associated with vaccination.

### Statistical Analysis

Statistical analysis was performed using GraphPad Prism software (version 9.0). HI titers and chicken weights were compared between experimental groups using one-way analysis of variance (ANOVA) followed by post-hoc Tukey's multiple comparison test. P-values less than 0.05 were considered statistically significant.

### Ethical Considerations

All animal experiments were conducted in accordance with the guidelines and regulations set forth by the Institutional Animal Care and Use Committee (IACUC). All efforts were made to minimize animal suffering and ensure the ethical treatment of experimental animals throughout the study.

### Results

Vaccine Design and Nanoadjuvant Characterization

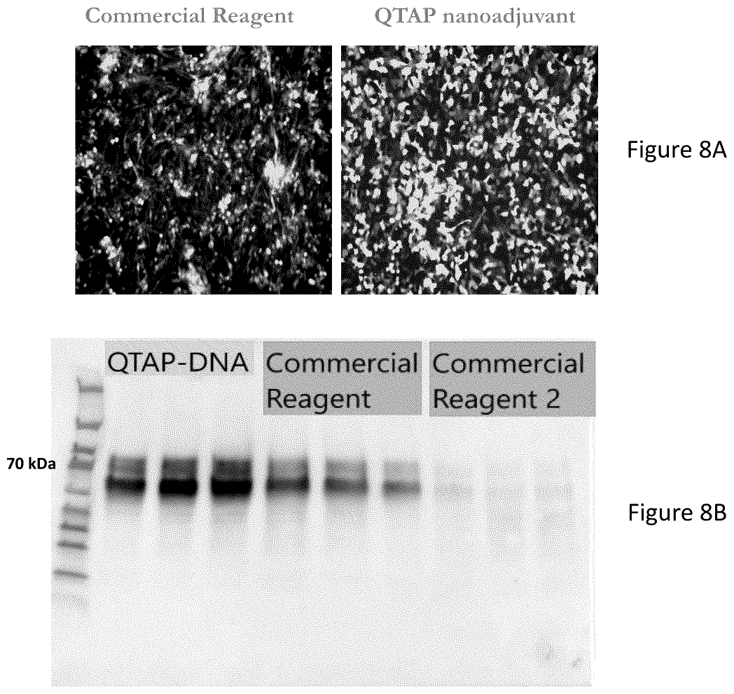

The mosaic DNA vaccine targeting conserved regions of Avian influenza virus (AIV) was successfully synthesized and cloned into a plasmid vector under the control of CMV early enhancer/chicken β actin (CAG) promoter. To assess the expression of the vaccine construct, BHK (Baby Hamster Kidney) cells were transfected with either the plasmid DNA encoding the mosaic DNA vaccine or GFP encoded Plasmid using the QTAP nanoadjuvant compared to a commercial transfection reagent. Fluorescence image acquired to visually determine GFP expression (FIG. 8A). Following transfection, cell lysates were harvested, and the expression of the vaccine antigen was evaluated by Western blot analysis using HA specific antibodies (FIG. 8B). The results confirmed the expression of the mosaic DNA vaccine in transfected BHK cells, demonstrating the successful delivery and expression of the vaccine construct in a mammalian cell line.

The nanoadjuvant was synthesized and characterized, demonstrating a mean particle size of 200 nm, a zeta potential of −25 mV, and a spherical morphology as determined by dynamic light scattering (DLS), zeta potential analysis, and transmission electron microscopy (TEM), respectively.

### Safety Assessment

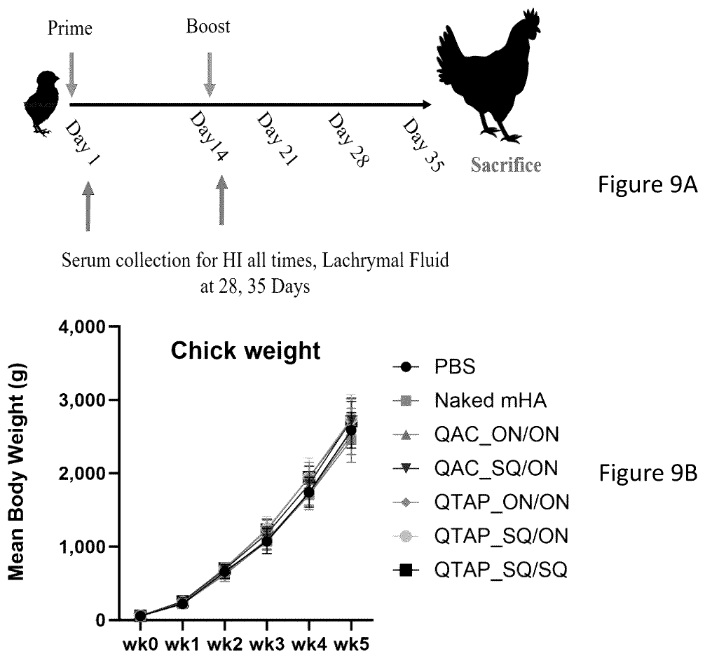

No significant changes in chicken weight were observed between the vaccinated and control groups throughout the study period (FIG. 9), suggesting that the nanoadjuvanted mosaic DNA vaccine did not adversely affect the growth rates or overall health of the immunized chickens. Immunogenicity of the Nanoadjuvanted Mosaic DNA Vaccine

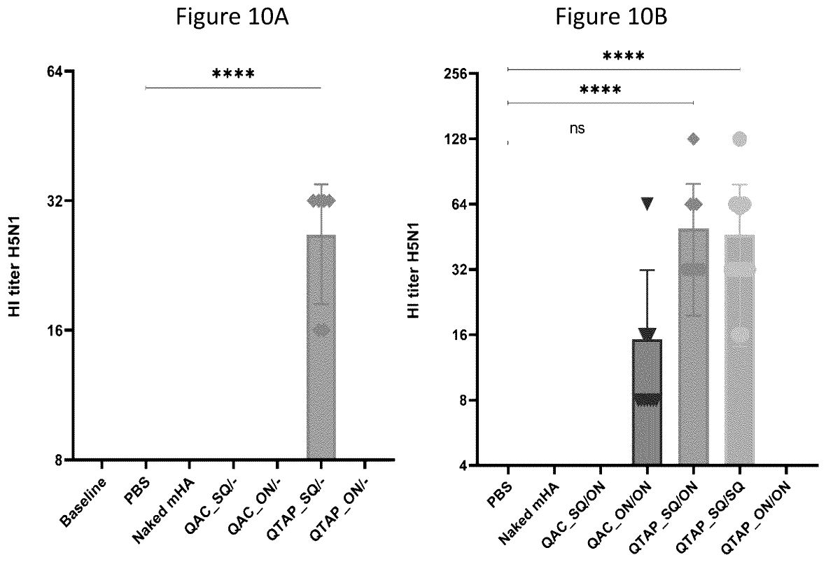

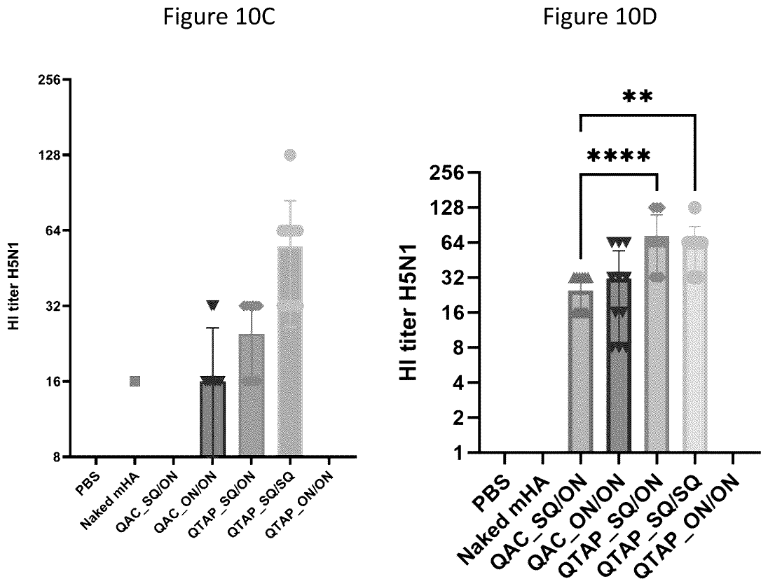

Chickens immunized with QTAP nanoadjuvanted mosaic DNA vaccine exhibited a significant increase in hemagglutination inhibition (HI) titers compared to control groups. Specifically, HI titers were observed to range from 1:32 to 1:128 against heterologous AIV strains, indicating a robust immune response elicited by the vaccine formulation (FIG. 10).

### Statistical Analysis

Statistical analysis revealed significant differences in HI titers between the vaccinated and control groups (P<0.05) for both homologous and heterologous AIV strains. However, no significant differences in chicken weight were observed between experimental groups, indicating the safety of the vaccine formulation.

### Discussion

The results of this study demonstrate the successful development and immunogenicity of a nanoadjuvanted mosaic DNA vaccine targeting Avian influenza virus (AIV) in chickens. The significant increase in HI titers following immunization suggests that the vaccine formulation effectively induced a specific and robust immune response against diverse AIV strains. Importantly, the absence of any significant changes in chicken weight throughout the study period indicates that the vaccine formulation maintained a favorable safety profile, further supporting its potential for use in commercial poultry production.

The incorporation of a novel nanoadjuvant in the vaccine formulation likely contributed to the enhanced immunogenicity observed, highlighting the importance of adjuvant selection and formulation optimization in vaccine development. Future studies will focus on evaluating the protective efficacy, duration of immunity, and potential for commercial application of the nanoadjuvanted mosaic DNA vaccine in poultry populations, thereby contributing to the ongoing efforts to control and prevent AIV outbreaks and mitigate the associated economic and zoonotic risks.

### References for Example 2

1. Peiris J S, de Jong M D, Guan Y. Avian influenza virus (H5N1): a threat to human health. Clin Microbiol Rev. 2007; 20 (2): 243-67; doi: 10.1128/CMR.00037-06.
2. Rehman S, Effendi M H, Witaningruma A M, Nnabuikeb U E, Bilal M, Abbas A, et al. Avian influenza (H5N1) virus, epidemiology and its effects on backyard poultry in Indonesia: a review. F1000Res. 2022; 11:1321; doi: 10.12688/f1000research. 125878.2.
3. Byrne A M P, James J, Mollett B C, Meyer S M, Lewis T, Czepiel M, et al. Investigating the Genetic Diversity of H5 Avian Influenza Viruses in the United Kingdom from 2020-2022. Microbiol Spectr. 2023; 11 (4): e0477622; doi: 10.1128/spectrum.04776-22.
4. Mo I P, Bae Y J, Lee S B, Mo J S, Oh K H, Shin J H, et al. Review of Avian Influenza Outbreaks in South Korea from 1996 to 2014. Avian Dis. 2016; 60 (1 Suppl): 172-7; doi: 10.1637/11095-041715-Review.
5. Wen X, Sun S, Li L, He Q, Tsai F S. Avian Influenza-Factors Affecting Consumers' Purchase Intentions toward Poultry Products. Int J Environ Res Public Health. 2019; 16 (21); doi: 10.3390/ijerph16214139.
6. Ly H. Highly pathogenic avian influenza H5N1 virus infection of companion animals. Virulence. 2024; 15 (1): 2289780; doi: 10.1080/21505594.2023.2289780.
7. Wang D, Zhu W, Yang L, Shu Y. The Epidemiology, Virology, and Pathogenicity of Human Infections with Avian Influenza Viruses. Cold Spring Harb Perspect Med. 2021; 11 (4); doi: 10.1101/cshperspect.a038620.
8. Lim C M L, Komarasamy T V, Adnan N, Radhakrishnan A K, Balasubramaniam V. Recent Advances, Approaches and Challenges in the Development of Universal Influenza Vaccines. Influenza Other Respir Viruses. 2024; 18 (3): e13276; doi: 10.1111/irv.13276.
9. Kim J H, Jacob J. DNA vaccines against influenza viruses. Curr Top Microbiol Immunol. 2009; 333:197-210; doi: 10.1007/978-3-540-92165-3_10.
10. Choo A Y, Broderick K E, Kim J J, Sardesai N Y. DNA-based influenza vaccines: evaluating their potential to provide universal protection. IDrugs. 2010; 13 (10): 707-12.
11. Cui J, O'Connell C M, Hagen C, Sawicki K, Smyth J A, Verardi P H, et al. Broad Protection of Pigs against Heterologous PRRSV Strains by a GP5-Mosaic DNA Vaccine Prime/GP5-Mosaic rVaccinia (VACV) Vaccine Boost. Vaccines (Basel). 2020; 8 (1); doi: 10.3390/vaccines8010106.
12. Cui J, O'Connell C M, Costa A, Pan Y, Smyth J A, Verardi P H, et al. A PRRSV GP5-Mosaic vaccine: Protection of pigs from challenge and ex vivo detection of IFNgamma responses against several genotype strains. PLOS One. 2019; 14 (1): e0208801; doi: 10.1371/journal.pone.0208801.
13. Stachyra A, Gora-Sochacka A, Sirko A. DNA vaccines against influenza. Acta Biochim Pol. 2014; 61 (3): 515-22.
14. Eusebio D, Neves A R, Costa D, Biswas S, Alves G, Cui Z, et al. Methods to improve the immunogenicity of plasmid DNA vaccines. Drug Discov Today. 2021; 26 (11): 2575-92; doi: 10.1016/j.drudis.2021.06.008.
15. Ma Q, Mu Y, Gong L, Zhu C, Di S, Cheng M, et al. Manganese-based nanoadjuvants for enhancement of immune effect of DNA vaccines. Front Bioeng Biotechnol. 2022; 10:1053872; doi: 10.3389/fbioe.2022.1053872.
16. Cao W, Kim J H, Reber A J, Hoelscher M, Belser J A, Lu X, et al. Nasal delivery of Protollin-adjuvanted H5N1 vaccine induces enhanced systemic as well as mucosal immunity in mice. Vaccine. 2017; 35 (25): 3318-25; doi: 10.1016/j.vaccine.2017.05.004.
17. Touray B J B, Hanafy M, Phanse Y, Hildebrand R, Talaat A M. Protective RNA nanovaccines against Mycobacterium avium subspecies hominissuis. Front Immunol. 2023; 14:1188754; doi: 10.3389/fimmu.2023.1188754.

We claim:

1. A composition comprising disaggregated spherical nanostructures comprising Quil-A and 1,2-dioleoyl-3-trimethylammonium propane (DOTAP) wherein the Quil-A and DOTAP are present at ratios between 2:1 Quil-A: DOTAP to about 1:2 Quil-A: DOTAP.

2. The composition of claim 1, wherein the disaggregated spherical nanostructures comprise a payload molecule.

3. The composition of claim 2, wherein the payload molecule is selected from the group consisting of a DNA molecule, an RNA molecule, a polynucleotide, a protein, a polypeptide, a pathogen, an attenuated pathogen, a small molecule, and antibody or combinations thereof.

4. The composition of claim 3, wherein the payload is a virus or bacteria, a viral or bacterial antigen, or a polynucleotide encoding a viral or bacterial antigen.

5. The composition of claim 3, wherein the payload is an mRNA molecule.

6. The composition of claim 2, wherein the payload molecule is an antigen derived from Infectious Bronchitis Virus (IBV), Severe Acute Respiratory Syndrome Coronavirus 2 (SARS-CoV-2), influenza virus, Mycobacterium avium subspecies paratuberculosis, Mycobacterium bovis, Mycobacterium tuberculosis, Mycobacterium avium subspecies avium, or Mycobacterium avium subspecies hominissuis.

7. The composition of claim 1, wherein the nanostructures are between about 50 nm and 100 nm in diameter in the absence of a payload molecule.

8. The composition of claim 1, wherein the composition is stable at any temperature for at least about 72 hours or stable for up to about 3 weeks at any temperature less than 4° C. with a payload molecule.

9. A method of forming a composition comprising Quil-A and DOTAP spherical nanostructures, comprising the steps of:
(i) Diluting Quil-A in a buffer, wherein Quil-A is present at a final concentration of about 0.02% to about 0.001%;
(ii) diluting DOTAP in a buffer in a separate container from Quil-A, wherein DOTAP is present at a final concentration of about 0.1% to about 0.008%;
(iii) combining the contents of (i) with the contents of (ii) and vortexing the combination for at least 15 seconds; and
(iv) incubating the combined vortexed solution of (iii) whereby a composition comprising Quil-A and DOTAP nanostructures are formed.

10. The method of claim 9, wherein the buffer of step (i) additionally comprises a payload molecule.

11. The method of claim 10, wherein the payload is an mRNA molecule.

12. The method of claim 10, wherein the Quil-A is diluted in step (i) to a final concentration of about 0.002% and DTOP is diluted in step ii to a final concentration of about 0.016%.

13. A composition comprising Quil-A and DOTAP nanostructures produced by the method of claim 9.

14. A vaccine formulation comprising: the composition of claim 1 and a pharmaceutically acceptable carrier.

15. A method of eliciting an immune response in a subject, the method comprising administering to the subject the composition of claim 1.

16. The method of claim 15, wherein the method induces a humoral and/or T-cell mediated immune response in the subject.

17. The method of claim 15, wherein when the composition comprises an mRNA payload molecule, the mRNA payload molecule can be detected up to 30 days after administering the composition to the subject.

18. A method of delivering a payload inside a cell, comprising contacting the cell with the composition of claim 1.

19. The method of claim 18, wherein the cells are contacted in vitro.

20. The method of claim 18, wherein the payload comprises a nucleotide or nucleoprotein complex.
